(19) United States
(12) Patent Application Publication
(10) Pub. No.: US 2015/0202321 A1
(43) Pub. Date: Jul. 23, 2015

(54) COMPOSITIONS COMPRISING CHITOSAN-DRUG CONJUGATES AND METHODS OF MAKING AND USING THE SAME

(71) Applicants: Farhan J. AHMAD, New Dehli (IN); Guarav K. JAIN, New Dehli (IN); Navdeep JAIKARIA, Titusville, NJ (US); NEWGEN BIOPHARMA CORP., Titusville, NJ (US)

(72) Inventors: Abu Alam, Lake Forest, IL (US); Navdeep Jaikaria, Titusville, NJ (US); Gaurav K. Jain, New Delhi (IN); Farhan J. Ahmad, New Delhi (IN)

(73) Assignee: NEWGEN BIOPHARMA CORP., Titusville, NJ (US)

(21) Appl. No.: 14/421,737

(22) PCT Filed: Aug. 14, 2013
(86) PCT No.: PCT/US13/54885

§ 371 Date: Feb. 13, 2015

Related U.S. Application Data
(60) Provisional application No. 61/683,184, filed on Aug. 14, 2012.

(51) Int. Cl. A61K 47/48; A61K 31/216; A61K 45/06; A61K 31/40

(52) U.S. Cl. CPC A61K 47/48869; A61K 47/4823; A61K 31/40; A61K 31/216; A61K 45/06

(57) ABSTRACT

The present disclosure relates to nanosized chitosan-statin conjugates, nanosized chitosan-chemotherapeutic agent conjugates, compositions comprising such nanosized chitosan-drug conjugates, and methods of making and using the same. The compositions result in unexpected and dramatic improved bioavailability of the component statin or chemotherapeutic agent.

### CROSS-REFERENCE TO RELATED APPLICATION

The present application claims priority to U.S. Provisional Application No. 61/683,184, filed on Aug. 14, 2012, which is specifically incorporated by reference.

### FIELD OF THE INVENTION

The present disclosure relates to nanosized chitosan-drug conjugates, compositions comprising such nanosized chitosan-drug conjugates, and methods of making and using the same. The drug present in the chitosan-drug conjugate can be a statin, chemotherapeutic agent, antibiotic, antifungal, or an asthma drug. The compositions result in unexpected and dramatic improved bioavailability of the component drug.

### BACKGROUND OF THE INVENTION

### I. Background Regarding Drug Delivery

Ease of active pharmaceutical ingredient delivery is a key issue facing pharmaceutical companies that develop and attempt to commercialize therapeutic products. An active pharmaceutical ingredient (API) that is readily soluble in water, for example, is not difficult to formulate into a suitable dosage form. However, formulating poorly water-soluble therapeutic drugs into suitable dosage forms poses a significant challenge. This is because the human body is a water based system; thus, as a condition of producing therapeutic activity, a drug must dissolve following administration.

Some poorly water-soluble API are never commercialized because they cannot be effectively solubilized, and therefore fail to exhibit acceptable in vivo therapeutic activity. Alternatively, the quantity of poorly water-soluble API required to be administered to achieve an acceptable level of therapeutic activity may be too great, given the poor water solubility of the agent, and result in unacceptable toxicity. Even if an API is formulated into a liquid, wherein the API is solubilized in a solvent, such dosage forms sometimes perform sub-optimally. For example, such dosage forms may have unpredictable properties or induce undesirable side effects. For example, Cremophor, which is a solvent used to solubilize active agents such as paclitaxel, can induce severe adverse allergic reaction in subjects, and resulting in death.

Prior art methods exist for enhancing API solubility. For example, the particle size of the API can be reduced, thereby increasing the exposed surface area of the API, resulting in greater water solubility. One prior method for particle size reduction is wet milling. This method requires grinding of an API with beads made of hard glass, porcelain, zirconium oxide, polymeric resin, or other suitable substance in a media in which the API is poorly soluble, such as water. The API is physically converted into much smaller particles that remain suspended in the grinding media. The resultant micron- or nanometer-sized API particles can then be isolated from the grinding media by methods such as by filtration or centrifugation, and formulated into an appropriate dosage form. See U.S. Pat. No. 5,145,684 for “Surface Modified Drug Nanoparticles;” U.S. Pat. Nos. 5,518,187 and 5,862,999, both for “Method of Grinding Pharmaceutical Substances;” and U.S. Pat. No. 5,718,388, for “Continuous Method of Grinding Pharmaceutical Substances.” The media in which the API is ground typically contains one or more compounds that function as a surface stabilizer for the API. The surface stabilizers adsorb to the surface of the API and act as a steric barrier to API particle size growth.

Conventional wet milling techniques therefore produce a “bi-phasic” system in which the stabilized API nanoparticles are suspended in the aqueous grinding media. The nanoparticulate drug delivery technology commercialized by Elan Drug Delivery (King of Prussia, Pa.) under the trade name NanoCrystal® technology, and SkyePharma, plc's Insoluble Drug Delivery (IDD®) technology exemplify such wet milling techniques. Four commercially marketed drugs are made utilizing Elan's NanoCrystal technology (EMEND®, RAPAMUNE®, TRICOR®, and MEGACE ES®), and TRIGLIDE® is made using SkyePharma's technology.

However, wet milling of API has drawbacks, principally being the cost of the process. The added cost for formulating a poorly water-soluble API into a nanoparticulate composition utilizing wet milling can be prohibitive.

Other known methods of making nanoparticulate active agent compositions include precipitation, homogenization, and super critical fluid methods. Microprecipitation is a method of preparing stable dispersions of poorly soluble API. Such a method comprises dissolving an API in a solvent followed by precipitating the API out of solution. Homogenization is a technique that does not use milling media. API in a liquid media constitutes a process stream propelled into a process zone, which in a Microfluidizer® (Microfluidic, Inc.) is called the Interaction Chamber. The geometry of the interaction chamber produces powerful forces of sheer, impact, and cavitation which are responsible for particle size reduction. U.S. Pat. No. 5,510,118 refers to a bi-phasic process using a Microfluidizer® resulting in nanoparticulate active agent particles. Finally, supercritical fluid methods of making nanoparticulate API compositions comprise dissolving an API in a solution. The solution and a supercritical fluid are then co-introduced into a particle formation vessel. The temperature and pressure are controlled, such that dispersion and extraction of the vehicle occur substantially simultaneously by the action of the supercritical fluid. Examples of known supercritical methods of making nanoparticles include International Patent Application No. WO 97/14407 and U.S. Pat. No. 6,406,718.

Polymer-drug conjugates as a type of drug delivery system have attracted attention due to their particular therapeutic properties, such as prolonged half-life, enhanced bioavailability, and often targeting to specific cells, tissues or organs by attaching a homing device. Drug-polymer conjugates often aim to increase the surface area, solubility and wettability of the powder particles and are therefore focused on particle size reduction or generation of amorphous states. Grau et al., Int. J. Pharm., 196: 155-159 (2000); Hancock and Zografi, J. Pharm. Sci., 86: 1-12 (1997); Lee et al., J. Control. Release, 140, 79-85 (2009); Yang et al., Bioorg. Med. Chem., 18: 117-123 (2010). Examples of polymer-drug conjugates include PHEA-50-O-succinyl zidovudine with a prolonged duration of activity (Giammona et al., J. Control. Release, 54L 321-331 (1998)). and the macromolecular prodrug of 3TC-dextran for selective antiviral delivery to the liver. Chimalakonda et al., Biocon. Chem., 18: 2097-2108 (2007). Very recently, it has been reported that paclitaxel conjugate with low molecular weight chitosan exhibited favorable features for oral delivery including: (1) increased water solubility of paclitaxel, (2) prolonged retention of the conjugate in the GI tract, (3) ability to bypass the P-glycoprotein mediated efflux, and (4) ability to bypass cytochrome P450-mediated metabolism, all of which led to enhanced bioavailability and antitumor efficacy in vivo. Lee et al., J. Med. Chem., 51: 6442-6449 (2008).

Two classes of drugs characterized by poor bioavailability corresponding to poor water-solubility of the drug include statins and chemotherapeutic agents.

An example of a chitosan conjugate is described in Yousefpour et al., Int. J. of Nanomedicine, 2011:1977-1990 (2011), which describes chitosan-doxorubicin conjugation carried out using succinic anhydride as a crosslinker. The antibody trastuzumab was then conjugated to the chitosan-doxorubin conjugate particles via thiolation of lysine residues and subsequent linking of the resulted thiols to chitosan. The reference does not teach or suggest size reduction of the chitosan conjugate using, for example, milling or any other size reduction process.

A summary of the use of chitosan as a drug delivery vehicle is also provided in Patel et al., J. Pharm. Pharmaceutical Sci., 13(3):536-557 (2010). This reference does not teach or suggest size reduction of chitosan conjugates.

Anwar et al., Eur. J. Pharmaceutical Sci., 44(3):241-249 (Oct. 9, 2011), describes constructing an amorphous nano-sized polymer-atorvastatin conjugate by an amide coupling reaction, followed by high pressure homogenization to produce particles with a mean size of 215 nm. The authors Gaurav K. Jain and Farhan J. Ahmad of this reference are co-inventors of the present application.

### II. Background Regarding Statins

A number of new drugs collectively known as statins or vastatins have been introduced to reduce serum LDL cholesterol levels, and representative examples of these drugs are detailed in The Merck Index. High LDL cholesterol levels have been shown to be an important risk factor in the development of arteriosclerosis and ischaemic heart disease. Statins have been found to lower serum LDL cholesterol levels in a dose dependent manner. Additionally, these drugs lower serum triglyceride levels, which is another risk factor for heart disease.

Statins lower serum LDL cholesterol levels by competitive inhibition of 3-hydroxyl-3-methylglutaryl-Coenzyme A reductase (HMG-COA reductase), an enzyme involved in the biosynthesis of cholesterol. By binding tightly to the active site of the enzyme, statins block the reduction of HMG-CoA, a step necessary in the biosynthesis of cholesterol. This inhibition of cholesterol biosynthesis by a statin results in a decrease in the production and secretion of LDL cholesterol. In addition, the upregulation of LDL receptors, especially in the liver, leads to the removal of LDLs from the serum. Thus, by reducing the production of LDL cholesterol and by causing LDL cholesterol to be removed from the serum, statins effectively reduce overall serum LDL cholesterol levels.

Two-thirds of the total cholesterol found in the body is of endogenous origin. The major site of cholesterol biosynthesis is in the liver. Such liver-derived cholesterol is the main cause of the development of hyper-cholesterolaemia. In contrast, cholesterol production in non-hepatic cells is needed for normal cell function. Therefore, selective inhibition of HMG-CoA reductase in the liver is an important requirement for HMG-COA reductase inhibitors. One of the problems with statin formulation is that currently available statins generally possess a low systemic bioavailability coupled with extensive first pass hepatic metabolism. Singla et al., J. of Pharm. Sci. and Tech., 1(2):84-87 (2009).

Atorvastatin is an exemplary statin. Atorvastatin ([R—(R/,R/)]-2-(4-fluorophenyl)-b,d-dihydroxy-5-(1-methylethyl)-3-phenyl-4-[(phenylamino) carbonyl]-1H-pyrrole-1-heptanoic acid, calcium salt (2:1) trihydrate), is a statin used for lowering blood cholesterol levels. Atorvastatin (AT) is an orally administered drug used for the treatment of elevated total cholesterol, low density lipoprotein and triglycerides, and to elevate high density lipoprotein cholesterol. It also stabilizes plaque and prevents strokes through anti-inflammatory and other mechanisms. Like all statins, AT works by selectively inhibiting HMG-CoA reductase, an enzyme that is involved in the biosynthesis of cholesterol. AT is a BCS class II drug, insoluble in aqueous solutions of pH 4, very slightly soluble in distilled water and pH 7.4 phosphate buffer, and has high intestinal permeability. AT is rapidly absorbed after oral administration, with time to reach peak concentrations (tmax) within 1-2 h but possess poor oral bioavailability (˜12%). Corsini et al., Pharmcol. Ther., 84: 413-428 (1999). The poor oral bioavailability is attributed to its low aqueous solubility, crystalline nature, and high hepatic first-pass metabolism. Lennernas, Clin. Pharmacokinet., 42:1141-1160 (2003). Furthermore, the bioavailability of AT is highly variable due to its instability in the acidic milieu of the stomach. Shah et al., Rapid Commun. Mass Spectrum., 22, 613-622 (2008). Poor oral bioavailability of AT results in administration of its high doses and engenders dose related undesirable adverse effects such as liver abnormalities, rhabdomyolysis, arthralgia, and kidney failure. There are many existing factors limiting the successful use of orally administered AT, including problems with drug formulation due to poor aqueous solubility and more importantly, insufficient and fluctuating bioavailability obtained after oral administration (Kim et al., Int. J. Pharm., 359: 211-219 (2008); Kim et al., Eur. J. Pharm. Biopharm., 69: 454-465 (2008)). Therefore, a novel approach is needed to resolve both the solubility and absorption issues related to statins such as AT.

A number of methods have been developed to improve the oral bioavailability of statins such as AT based on improving the solubility and enhancing dissolution rate of the drug. For instance, it was reported that the use of self-microemulsifying drug delivery system (SMEDDS) for the delivery of a statin such as AT could improve the drug's solubility and permeability through the mucous membrane significantly. Shen and Zhong, J. Pharm. Pharmacol., 58:1183-1191 (2006). More recently, it has been reported that the solubility and bioavailability of crystalline AT could be improved by physical modification such as particle size reduction and conversion to amorphous state. Kim et al., Int. J. Pharm., 359: 211-219 (2008); Kim et al., Eur. J. Pharm. Biopharm., 69: 454-465 (2008); Zhang et al., Int. J. Pharm. 374: 106-113 (2009).

### III. Background Regarding Chemotherapeutic Agents

The majority of chemotherapeutic drugs can be divided in to alkylating agents, antimetabolites, anthracyclines, plant alkaloids, topoisomerase inhibitors, and other antitumor agents. All of these drugs affect cell division or DNA synthesis and function in some way.

Oral chemotherapy is a preferred alternative strategy in the cancer treatment due to its convenience, patient compliance and cost-effectiveness. However, the low oral bioavailability of anticancer drugs greatly limits the progress for oral cancer chemotherapy. Enhancement of oral bioavailability of anticancer drugs is a pre-requisite for successful development of oral modes of cancer treatment. While many anticancer agents are administered intravenously, again the low water solubility of many anticancer drugs limits their bioavailability and anticancer efficacy in vivo.

Dosage of chemotherapy can be difficult: If the dose is too low, it will be ineffective against the tumor, whereas, at excessive doses, the toxicity (side-effects, neutropenia) will be intolerable to the patient. Most chemotherapy is delivered intravenously, although a number of agents can be administered orally (e.g., melphalan, busulfan, capecitabine). Harmful and lethal toxicity from chemotherapy limits the dosage of chemotherapy that can be given. Some tumors can be destroyed by sufficiently high doses of chemotherapeutic agents. However, these high doses cannot be given because they would be fatal to the patient.

Chemotherapeutic techniques have a range of side-effects that depend on the type of medications used. The most common medications affect mainly the fast-dividing cells of the body, such as blood cells and the cells lining the mouth, stomach, and intestines. Common side-effects include: depression of the immune system, which can result in potentially fatal infections; fatigue; tendency to bleed easily; gastrointestinal distress (nausea and vomiting); hair loss; as well as damage to specific organs, including cardiotoxicity (heart damage), hepatotoxicity (liver damage), nephrotoxicity (kidney damage), ototoxicity (damage to the inner ear, producing vertigo), and encephalopathy (brain dysfunction).

A dosage form providing a higher bioavailability of a chemotherapeutic agent could enable the use of lower doses of drug, thereby decreasing toxicity and side effects, while simultaneously increasing the effectiveness of the drug.

### IV. Background Regarding Antibiotics

An antibacterial is an agent that inhibits bacterial growth or kills bacteria. The term is often used synonymously with the term antibiotic. Today, however, with increased knowledge of the causative agents of various infectious diseases, antibiotic denotes a broader range of antimicrobial compounds, including anti-fungal and other compounds.

Most of today's antibacterials chemically are semisynthetic modifications of various natural compounds. These include, for example, the beta-lactam antibacterials, which include the penicillins (produced by fungi in the genus Penicillium), the cephalosporins, and the carbapenems. Compounds that are still isolated from living organisms are the aminoglycosides, whereas other antibacterials—for example, the sulfonamides, the quinolones, oxazolidinones, and ripamfin—are produced solely by chemical synthesis. Antibacterials are divided into two broad groups according to their biological effect on microorganisms: bactericidal agents kill bacteria, and bacteriostatic agents slow down or stall bacterial growth.

Some antibacterials are associated with a range of adverse effects, from mild—such as a fever and/or nausea—to very serious—such as major allergic reactions, including photodermatitis and anaphylaxis. Common side-effects include diarrhea, resulting from disruption of the species composition in the intestinal flora, resulting, for example, in overgrowth of pathogenic bacteria, such as Clostridium difficile.

### IV. Background Regarding Asthma Drugs

Treatment with asthma medication focuses on controlling inflammation and preventing symptoms (controller medication) and easing asthma symptoms when a flare-up occurs (quick-relief medication). Controller medication is the most important type of therapy for most people with asthma because these asthma medications prevent asthma attacks on an ongoing basis. These drugs include steroids or corticosteroids, inhaled long-acting beta-agonists (LABAs), and leukotriene modifiers. As a result of controller medications, airways are less inflamed and less likely to react to triggers. Quick relief medications are also called rescue medications and consist of short-acting beta-agonists (SABA). They relieve the symptoms of asthma by relaxing the muscles that tighten around the airways.

There is a need in the art for improved methods of formulation active agents, such as statins, chemotherapeutic agents, antibiotics, and asthma drugs. The present invention satisfies this need.

### SUMMARY OF THE INVENTION

The claimed invention is directed to nanosized chitosan-drug conjugates and compositions comprising the same, wherein the drug is a statin, chemotherapeutic agent, antibiotic, angtifungal or asthma drug. Preferably the drug is poorly water-soluble. “Poorly water-soluble” generally means that the drug has a solubility in water of less than about 10 mg/mL, or in other embodiments less than about 5 mg/mL, or less than about 1 mg/mL. The nanosized chitosan-drug conjugates can have an average particle size of less than about 1000 nm. The compositions can further comprise one or more pharmaceutically acceptable excipients.

In one embodiment of the invention, encompassed is a composition comprising a nanosized chitosan-statin conjugate prepared according to the invention and methods of making and using the same. The methods of the invention comprise administering the nanosized chitosan-statin conjugate to a subject in need; i.e., a subject having high cholesterol levels and/or cancer. Exemplary statins include, but are not limited to, atorvastatin and rosuvastatin. Additional statins are described herein. In one embodiment, the statin is not atorvastatin. The composition can be administered via any pharmaceutically acceptable method, as described herein, including oral administration.

In another embodiment of the invention, encompassed is a composition comprising a nanosized chitosan-statin conjugate prepared according to the invention combined with a fenofibrate nanoemulsion composition and methods of making and using the same. In one embodiment, the statin is not atorvastatin. The methods of the invention comprise administering the composition comprising nanosized chitosan-statin conjugates and a fenofibrate nanoemulsion to a subject in need; i.e., a subject having high cholesterol levels and/or cancer. Exemplary statins include, but are not limited to, atorvastatin and rosuvastatin. Additional statins are described herein. Methods of making the nanoemulsion fenofibrate are described, for example, in US 2007/0264349. The composition can be administered via any pharmaceutically acceptable method, as described herein, including oral administration. The fenofibrate nanoemulsion comprises fenofibrate, at least one solvent, at least one surfactant, and at least one oil. Additionally, the fenofibrate nanoemulsion can comprises oil droplets having a droplet size of less than about 3 microns. The fenofibrate nanoemulsion can also comprise fenofibrate particles having an average particle size of less than about 3 microns. Furthermore, the fenofibrate nanoemulsion oil droplets can comprise solubilized fenofibrate, fenofibrate particles, or a combination thereof.

In yet another embodiment, encompassed is a composition comprising a nanosized chitosan-chemotherapeutic agent conjugate prepared according to the invention and methods of making and using the same. The methods of the invention comprise administering the nanosized chitosan-chemotherapeutic agent conjugate to a subject in need; i.e., a subject having a cancer and/or in need of chemotherapeutic treatment. Exemplary chemotherapeutic agents include, but are not limited to, paclitaxel and docetaxel. Additional chemotherapeutic agents are described herein. The composition can be administered via any pharmaceutically acceptable method, as described herein, including oral administration. In one embodiment, a composition comprising the nanosized chitosan-chemotherapeutic agent conjugate is sterile and administered parenterally (IM/IV/peritoneal). In another embodiment, the composition comprising the nanosized chitosan-chemotherapeutic agent conjugate is lyophilized, followed by reconstitution with a suitable vehicle for parenteral administration.

In another embodiment, encompassed is a composition comprising a nanosized chitosan-antibiotic or antifungal conjugate prepared according to the invention and methods of making and using the same. The methods of the invention comprise administering the nanosized chitosan-antibiotic or antifungal conjugate to a subject in need; i.e., a subject having a microbial or antifungal infection and/or in need of antimicrobial or antimicrobial treatment. Examples of antibiotics are described herein. The composition can be administered via any pharmaceutically acceptable method, as described herein, including oral, injectable, inhalation, topical, etc.

In another embodiment, encompassed is a composition comprising a nanosized chitosan-asthma drug conjugate prepared according to the invention and methods of making and using the same. The methods of the invention comprise administering the nanosized chitosan-asthma drug conjugate to a subject in need; i.e., a subject having asthma and/or in need of asthma treatment. Examples of asthma drugs are described herein. The composition can be administered via any pharmaceutically acceptable method, as described herein, including oral, nasal, injectable, inhalation, topical, etc.

In another embodiment of the invention, the nanosized chitosan-drug conjugates of the invention can be formed using an amide coupling reaction between the amine groups of chitosan and an activated group, such as an activated carboxylic group, of the statin, chemotherapeutic agent, antibiotic, antifungal or asthma drug. The resultant conjugate can comprise an amide linker that is cleaved under physiological conditions.

In one embodiment of the invention, the nanosized chitosan-drug conjugates demonstrate an increase in water solubility of the component drug as compared to a non-nanosized chitosan conjugate of the same drug, present at the same dosage. In another embodiment, the nanosized chitosan-drug conjugates demonstrate an increase in bioavailability of the component drug as compared to a non-nanosized chitosan conjugate of the same drug, present at the same dosage.

In yet another embodiment of the invention, the nanosized chitosan-drug conjugates demonstrate an increase in mucoadhesion as compared to a non-nanosized chitosan conjugate dosage form of the same drug, present at the same dosage.

In yet another embodiment of the invention, the nanosized chitosan-drug conjugates prevent the degradation of the drug in the acidic milieu of the stomach.

In one embodiment of the invention, the compositions of the invention comprising a nanosized chitosan-drug conjugate exhibit improved pK profiles for the component drug. For example, the compositions of the invention can exhibit an improved phrarmacokinetic parameters when administered orally such as Tₘₐₓ, Cₘₐₓ, AUC, or any combination thereof. Specifically, in the compositions of the invention (1) the Tₘₐₓ of the statin, chemotherapeutic agent, antibiotic, antifungal or asthma drug, when assayed in the plasma of a mammalian subject following administration, can be less than the Tₘₐₓ for a conventional, non-chitosan nanosized conjugate form of the same statin, chemotherapeutic agent, antibiotic, antifungal or asthma drug, administered at the same dosage; (2) the Cₘₐₓ of the statin, chemotherapeutic agent, antibiotic, antifungal or asthma drug, when assayed in the plasma of a mammalian subject following administration, can be greater than the Cₘₐₓ for a conventional, non-chitosan nanosized conjugate form of the same statin, chemotherapeutic agent, antibiotic, antifungal or asthma drug, administered at the same dosage; and/or (3) the AUC of the statin, chemotherapeutic agent, antibiotic, antifungal or asthma drug, when assayed in the plasma of a mammalian subject following administration, is greater than the AUC for a non-chitosan nanosized conjugate form of the same statin, chemotherapeutic agent, antibiotic, antifungal or asthma drug, administered at the same dosage.

Furthermore, in another embodiment, the pharmacokinetic profile of the statin, chemotherapeutic agent, antibiotic, antifungal or asthma drug present in the nanosized chitosan-conjugates of the invention is not substantially affected by the fed or fasted state of a subject ingesting the composition, when administered to a human.

Also encompassed by the invention are methods for treating or preventing dyslipidemia, hyperlipidemia, hypercholesterolemia, cardiovascular disorders, hypertriglyceridemia, coronary heart disease, peripheral vascular disease, symptomatic carotid artery disease), or related conditions comprising administering to a subject in need a composition comprising a nanosized chitosan-statin conjugate according to the invention. The composition comprising a nanosized chitosan-statin can further comprise a fenofibrate nanoemulsion.

Also encompassed by the invention are methods for reducing LDL-C, total-C, triglycerides, and/or Apo B in adult patients with primary hypercholesterolemia or mixed dyslipidemia (Fredrickson Types IIa and IIb), comprising administering to a subject in need a composition comprising a nanosized chitosan-statin conjugate according to the invention. In one embodiment, the statin is not atorvastatin. The composition comprising a nanosized chitosan-statin can further comprise a fenofibrate nanoemulsion.

The invention encompasses methods for treating adult patients with hypertriglyceridemia (Fredrickson Types IV and V hyperlipidemia) comprising administering to a subject in need a composition comprising a nanosized chitosan-statin conjugate according to the invention. The composition comprising a nanosized chitosan-statin can further comprise a fenofibrate nanoemulsion.

The invention encompasses methods for treating pancreatitis, restenosis, and Alzheimer's disease comprising administering to a subject in need a composition comprising a nanosized chitosan-statin conjugate according to the invention. The composition comprising a nanosized chitosan-statin can further comprise a fenofibrate nanoemulsion.

The invention encompasses methods for treating, preventing, and/or reducing the risk of a cancer comprising administering to a subject in need a composition comprising a nanosized chitosan-chemotherapeutic agent conjugate according to the invention, a nanosized chitosan-statin according to the invention, or, a nanosized chitosan-statin according to the invention in combination with a fenofibrate nanoemulsion. The cancer can be any cancer, including but not limited to a solid tumor or a hematopoietic disorder.

The invention encompasses methods for treating and/or preventing a microbial infection comprising administering to a subject in need a composition comprising a nanosized chitosan-antibiotic or antifungal conjugate according to the invention. The chitosan-antibiotic and/or chitosan-antifungal conjugates can be administered via any pharmaceutically acceptable means, including for example by inhalation with direct delivery into the lungs to maximize the concentration in the deep compartments of the lungs in patients suffering from lung diseases such as cystic fibrosis, pneumonia, tuberculin bacilli. Also, the nano chitosan-drug conjugates can be deposited into the deep compartments of lungs by inhalation resulting in rapid onset of action to counter bio-terrorism exposure to inhaled anthrax organism.

The invention encompasses methods for treating and/or preventing asthma symptoms comprising administering to a subject in need a composition comprising a nanosized chitosan-asthma drug conjugate according to the invention.

Also encompassed by the invention is a method of making a nanosized drug-chitosan conjugate, wherein the drug is a statin, chemotherapeutic agent, antibiotic, antifungal or asthma drug. In one embodiment, the statin is not atorvastatin. The method comprises activating a carboxylic group of the statin, chemotherapeutic agent, antibiotic, antifungal or asthma drug, followed by covalently attaching the statin, chemotherapeutic agent, antibiotic, antifungal or asthma drug to chitosan via an amide linker using an amide coupling reaction between amine groups of chitosan and the activated carboxylic group of the drug to obtain a chitosan-drug conjugate. The chitosan-drug conjugate is then homogenized to reduce the particle size of the chitosan-drug conjugate to less than about 1000 nm. The amide linker is preferably cleaved under physiological conditions. Additionally, the homogenization process is preferably a high pressure homogenization process. Finally, the chitosan-drug conjugates can be lyophilized or spray dried prior to or after the homogenization process. The method can further comprise adding a fenofibrate nanoemulsion to a chitosan-statin conjugate composition. In one embodiment, the fenofibrate nanoemulsion can be lyophilized or spray dried to form a powder prior to combining with chitosan-statin conjugate composition

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1: Shows a schematic diagram for preparation of chitosan-statin (atorvastatin; AT) nano-conjugate.

FIG. 2: Shows ¹H NMR spectrum of atorvastatin (FIG. 2A), chitosan (FIG. 2B), chitosan (CH)-atorvastatin (AT) conjugate (FIG. 2C), chitosan (CH)-atorvastatin (AT) nanoconjugate (FIG. 2D).

FIG. 3: Shows FT-IR spectra of AT (FIG. 3A), chitosan (FIG. 3B), CH-AT conjugate (FIG. 3C), CH-AT nano-conjugate (FIG. 3D).

FIG. 4: Shows SEM images of AT (FIG. 4A), chitosan (FIG. 4B), CH-AT conjugate (FIG. 4C), CH-AT nano-conjugate (FIG. 4D).

FIG. 5: Shows XRD pattern of AT (FIG. 5A), chitosan (FIG. 5B), CH-AT conjugate (FIG. 5C), CH-AT nano-conjugate (FIG. 5D).

FIG. 6: Shows acidic degradation kinetics of AT and CH-AT nano-conjugate in 1 N HCl at 80° C.

FIG. 7: Shows plasma AT concentration as a function of time after oral administration of aqueous dispersion of AT (FIG. 7A) and CH-AT nano-conjugate (FIG. 7B) to rats.

FIG. 8: Shows a schematic representation of possible mechanism of drug release and bioavailability enhancement of AT through chitosan-atorvastatin nano-complex.

### DETAILED DESCRIPTION OF THE PREFERRED EMBODIMENTS

### I. Overview of the Invention

The present application relates a novel approach to improve the bioavailability and stability of statins, chemotherapeutic agents, antibiotics, antifungals and asthma drugs. The method comprises constructing a polymer-drug conjugate through amide coupling reaction, followed by size reduction of the conjugate via homogenization to obtain a nanosized polymer-drug conjugate. The component drug is a statin, chemotherapeutic agent, antibiotic, antifungal or asthma drug. The nanosized chitosan-drug nanoconjugates demonstrate a dramatic increase in solubility and a corresponding increase in bioavailability of the component drug.

Chitosan is a hydrophilic water-soluble macromolecule with active amine-functional groups. It is mucoadhesive in nature and is also known to improve permeation of drug molecules across biological barriers. Robinson et al., Ann. NY Acad. Sci., 507: 307-314 (1987); Smart et al., J. Pharm. Pharmacol., 36: 295-299 (1984). On the other hand, AT is a hydrophobic drug consisting of free carboxylic group. Peppas and Buri, J. Control. Release, 2: 257-275 (1985). The complex between chitosan and a statin was attempted to try to impart hydrophilicity (increased water solubility of the statin by conjugation to water soluble chitosan) and mucoadhesion (prolonged retention of the conjugate in the GI tract) to the statin. Further, it was hypothesized that the conjugate would also be able to prevent the degradation of the drug (e.g., statin or chemotherapeutic agent) in the acidic milieu of the stomach.

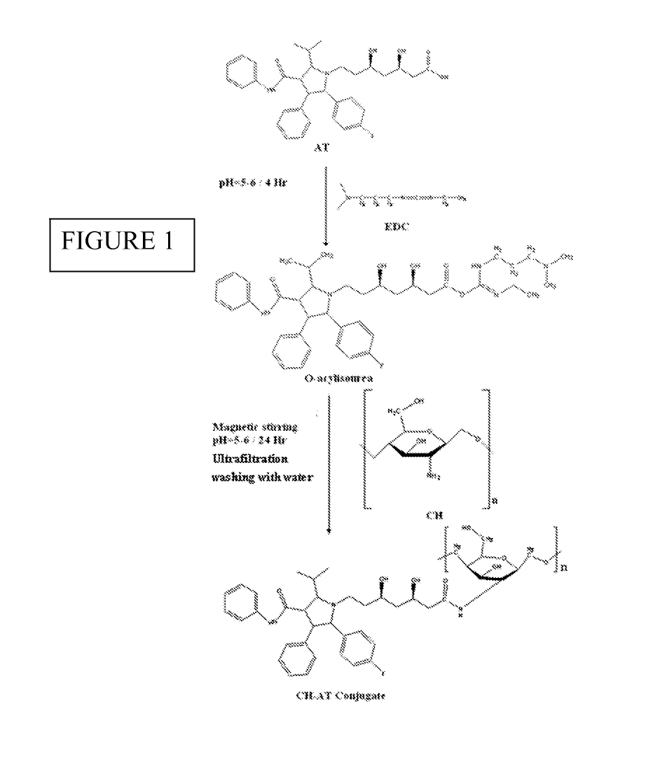

The chemical structure of a nanosized chitosan-drug (e.g., atorvastatin) conjugate is shown in FIG. 1. The drug was covalently attached to chitosan through an amide linker that is known to be cleaved under physiological conditions. Martin, Biopolymers, 45: 351-353 (1998); Testa, B., Biochem. Pharmacol., 68: 2097-2106 (2004). The conjugation between chitosan and the drug was carried out using an amide coupling reaction between the amine groups of chitosan and an activated carboxylic group of the statin (FIG. 1). Thus, preferably the drug (either statin or chemotherapeutic agent) present in the chitosan conjugate of the invention comprises a chemical group amenable to activation, such as a carboxylic group, to facilitate the conjugation method of the invention. The carboxylic group of the drug was activated using 1-Ethyl-3-(3-dimethyl aminopropyl)carbodiimide (EDC) by the formation of O-acylisourea, which could be viewed as a carboxylic ester with an activated leaving group (FIG. 1). EDC was selected because of its solubility in a wide range of solvents and easy separation of its by-product. EDC is a water soluble carbodiimide usually obtained as the hydrochloride and is generally used as a carboxyl activating agent for the coupling of primary amines to yield amide bonds. Other compounds can also be used to activate a carboxylic group to facilitate the conjugation between chitosan and a statin or chemotheraepeutic agent. See e.g., Montalbetti and Falque, Tetrahedron, 61:10827-10852 (2005). Alternatively, other methods and strategies known in the art for facilitating the formation of an amide bond can be used to make the conjugation product of the invention. See Montalbetti and Falque (2005).

Described herein is the synthesis of an exemplary nanosized chitosan-drug conjugate, where the drug is a statin, chemotherapeutic agent, antibiotic, antifungal or asthma drug, as well as the physicochemical characteristics and pharmacokinetics of the new prodrug. The chitosan-drug nanoconjugate detailed in the examples showed markedly enhanced water solubility (˜100 times) and better stability of the component statin in simulated gastric milieu. In vitro drug release studies indicate that the polymeric conjugate prodrug released the component drug for a prolonged period. Compared to suspension of the same drug (i.e., a statin), the chitosan-drug nanoconjugate exhibited less variable and 5-fold higher oral bioavailability. Taken together, chitosan-based conjugate system may be used as a delivery platform for poorly water-soluble statins, chemotherapeutic agents, antibiotics, antifungal and asthma drugs.

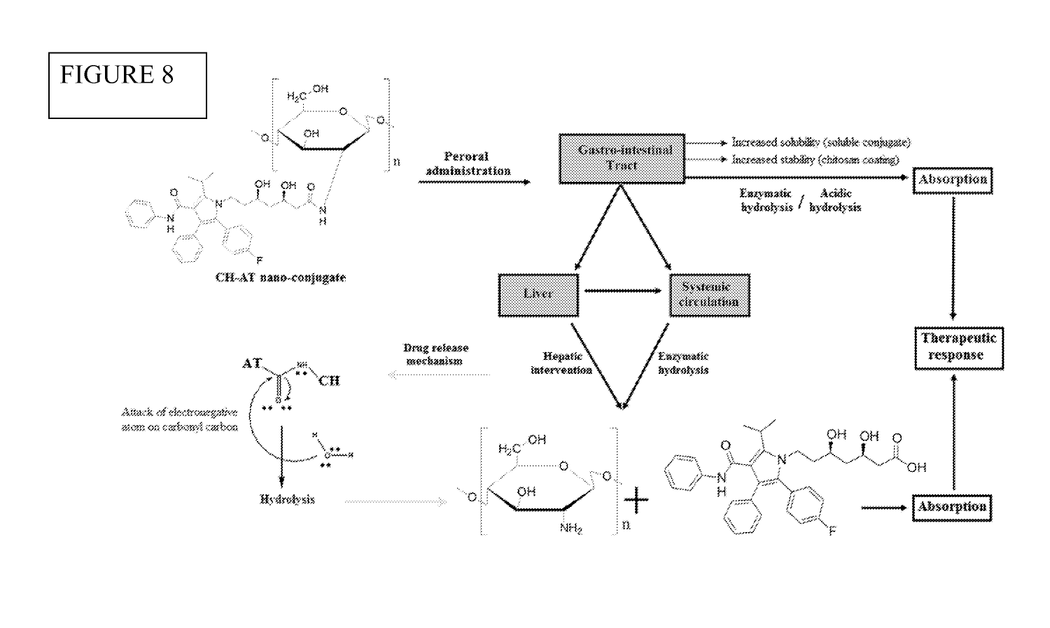

This unprecedented high absorption may be attributed to enhanced solubility of amorphous drug in the chitosan-drug nano-complex, and/or the known ability of chitosan to be mucoadhesive and open tight junctions in intestinal epithelial cells. Furthermore, the chitosan-drug nano-conjugate may also be able to bypass both P-glycoprotein-mediated efflux (displayed on intestinal epithelial cells) and cytochrome P450-mediated drug metabolism (hepatic clearance) as demonstrated previously for oral delivery of paclitaxel in the form of conjugate with chitosan. Lee et al., J. Med. Chem. 51: 6442-6449 (2008). The possible mechanism of drug release and bioavailability enhancement of AT through CH-AT nano-complex is depicted in FIG. 8.

Moreover, as detailed in the examples, the drug present in the nanosized chitosan-drug conjugate is released following in vivo administration. Specifically, the data in the Examples below teaches that the component drug is released from the nanosized conjugate under physiological conditions (Table 2). Furthermore, complete release of the component drug was obtained in simulated gastric fluid (SGF) within 6 hours. These results also suggest that the nanosized chitosan conjugate protects the component drug from acid catalyzed degradation.

The chitosan-antibiotic and/or chitosan-antifungal conjugates can be administered via any pharmaceutically acceptable means, including for example by inhalation with direct delivery into the lungs to maximize the concentration in the deep compartments of the lungs in patients suffering from lung diseases such as cystic fibrosis, pneumonia, tuberculin bacilli. Also, the nano chitosan-drug conjugates can be deposited into the deep compartments of lungs by inhalation resulting in rapid onset of action to counter bio-terrorism exposure to inhaled anthrax organism.

Direct delivery of active agents into the lungs can be particularly beneficial for treating various conditions. For example, delivery of active agents directly to the lungs (e.g., antifungals, antibiotics, asthma drugs) by inhalation can avoid systemic side effects associated with other target organs in patients suffering from various lung ailments, including e.g., AIDS.

Delivery of nanosized chitosan-drug conjugates directly into the lungs by inhalation is also beneficial as such a delivery method requires a fraction of the oral or parenteral drug dosage to obtain the desired therapeutic level of drug in the blood stream. Such a delivery method is also highly desirable when the disease site is localized in the lungs. For example, inhalation for site delivery into the deep lungs is optimal for a chitosan-chemotherapeutic agent conjugate for treating lung cancer, or a respiratory tumor. Another example is inhalation delivery for a chitosan-antibiotic or chitosan-antifungal conjugate for the treatment of cystic fibrosis or pneumonia or tuberculosis, an upper or lower respiratory tract infection, or anthrax poisoning. Yet another example is inhalation delivery for a chitosan-antifungal conjugate for the treatment of aspergillosis and mold present in the lungs. Finally, inhalation delivery of a chitosan-steroid or chitosan-asthma conjugate is useful in treating airway diseases such as chronic obstructive pulmonary disease (COPD) and asthma. As used herein, “asthma” encompasses all airway or respiratory diseases, including but not limited to COPD, conventional asthma, Inflammatory lung disease, Obstructive lung diseases, and Restrictive lung diseases.

In one embodiment of the invention, the drug dosage required to obtain the desired therapeutic effect, when delivery is via inhalation to the lungs of a chitosan-drug conjugate, is less than half that required to obtain the same therapeutic effect when the delivery route is oral or parenteral and the drug is not present in a chitosan conjugate. In other embodiments, the drug dosage required to obtain the desired therapeutic effect, when delivery is via inhalation to the lungs of a chitosan-drug conjugate, is about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, or about 3% of the drug dosage required to obtain the same therapeutic effect when the delivery route is oral or parenteral and the drug is not present in a chitosan conjugate.

The major types of respiratory system cancer are small cell lung cancer, non-small cell lung cancer, adenocarcinoma, large cell undifferentiated carcinoma, other lung cancers (carcinoid, Kaposi's sarcoma, melanoma), lymphoma, head and neck cancer, and mesothelioma, usually caused by exposure to asbestos dust, all of which can be treated using a nanosized chitosan-chemotherapeutic conjugate composition according to the invention.

Another benefit to targeted lung delivery is that since many cancers spread via the bloodstream and the entire cardiac output passes through the lungs, it is common for cancer metastases to occur within the lung. Breast cancer may invade directly through local spread, and through lymph node metastases. After metastasis to the liver, colon cancer frequently metastasizes to the lung. Prostate cancer, germ cell cancer and renal cell carcinoma may also metastasize to the lung. Thus, targeted lung delivery of a chemotherapeutic agent may provide better therapeutic results. This is significant as the chance of surviving lung cancer depends on the cancer stage at the time the cancer is diagnosed and is only about 14-17% overall. With current conventional treatment, in the case of metastases to the lung, treatment can occasionally be curative but only in certain, rare circumstances.

Furthermore, compositions comprising a nanosized chitosan-drug conjugate according to the invention can exhibit sustained release of the component drug. Such sustained release can be desirable for a statin, where a steady and consistent quantity of the drug in the bloodstream is desired to maintain optimal cholesterol levels. Similarly, such sustained and controlled release can be desirable for a chemotherapeutic agent, where a rapid release of a large amount of drug may result in more acute side effects and toxicity. Sustained release can also be desirable for an antibiotic or antifungal where a steady and consistent quantity of the drug in the bloodstream or lung tissue is desired to combat a microbial or fungal infection, and ineffective quantities of drug can result in resistant microbes. Finally, sustained release can also be desirable for an asthma drug, where insufficient quantity of drug present in the lung or bloodstream can result in an asthma attack (e.g., for controller medications). The sustained or controlled release of the drug from the nanosized chitosan-drug conjugate can be over a period of time, such as from about 2 to about 24 hours. In other embodiments of the invention, the sustained or controlled release of the drug from the chitosan-drug conjugate can be over a period of time such as about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

The present invention provides a method of prolonging plasma levels of a drug such as a statin, chemotherapeutic agent, antibiotic, antifungal or an asthma drug in a subject while achieving the desired therapeutic effect. In one aspect, such a method comprises orally administering to a subject an effective amount of a composition comprising a nanosized chitosan-drug conjugate according to the invention. In another aspect, such a method comprises administering to a subject via any pharmaceutically acceptable means an effective amount of a composition comprising a nanosized chitosan-drug conjugate according to the invention, including but not limited to pulmonary, inhalation, nasal, and injectable routes of administration.

In one embodiment of the invention, encompassed is a composition comprising a nanosized chitosan-statin conjugate prepared according to the invention and methods of making and using the same. The methods of the invention comprise administering the nanosized chitosan-statin conjugate to a subject in need, e.g., a subject in having high cholesterol levels. Exemplary statins include, but are not limited to, atorvastatin and rosuvastatin. Additional statins are described herein. The composition can be administered via any pharmaceutically acceptable method.

In another embodiment of the invention, encompassed is a composition comprising a nanosized chitosan-statin conjugate prepared according to the invention combined with a fenofibrate nanoemulsion composition and methods of making and using the same. As used herein, the term “statin compositions” or “nanosized chitosan-statin compositions” encompasses compositions comprising a nanosized chitosan-statin conjugate and additionally compositions comprising a nanosized chitosan-statin conjugate in combination with a fenofibrate nanoemulsion. The methods of the invention comprise administering the composition comprising nanosized chitosan-statin conjugates and a fenofibrate nanoemulsion to a subject in need; i.e., a subject having high cholesterol levels. Exemplary statins include, but are not limited to, atorvastatin and rosuvastatin. Additional statins are described herein. Methods of making the nanoemulsion fenofibrate are described, for example, in US 2007/0264349. The composition can be administered via any pharmaceutically acceptable method, as described herein, including oral administration. The fenofibrate nanoemulsion can be formulated into any pharmaceutically acceptable dosage form as described herein. For example, the fenofibrate nanoemulsion can be dried via a spray drying or lyophilization technique. The resultant dry powder fenofibrate nanoemulsion can then, for example, be blended with the chitosan-statin conjugate, followed by formulating the power blend into a capsule, tablet, or dosage form for reconstitution (e.g., suspension).

In yet another embodiment, encompassed is a composition comprising a nanosized chitosan-chemotherapeutic agent conjugate prepared according to the invention and methods of making and using the same. The methods of the invention comprise administering the nanosized chitosan-chemotherapeutic agent conjugate to a subject in need; i.e., a subject having a cancer and/or in need of chemotherapeutic treatment. Exemplary chemotherapeutic agents include, but are not limited to, paclitaxel and docetaxel. Additional chemotherapeutic agents are described herein. The composition can be administered via any pharmaceutically acceptable method, as described herein, including oral administration. In one embodiment, a composition comprising the nanosized chitosan-chemotherapeutic agent conjugate is sterile and administered parenterally (IM/IV/peritoneal). In another embodiment, the composition comprising the nanosized chitosan-chemotherapeutic agent conjugate is lyophilized, followed by reconstitution with a suitable vehicle for parenteral administration.

In yet another embodiment, encompassed is a composition comprising a nanosized chitosan-antibiotic or antifungal conjugate prepared according to the invention and methods of making and using the same. The methods of the invention comprise administering the nanosized chitosan-antibiotic or antifungal conjugate to a subject in need; i.e., a subject having a microbial or antifungal infection and/or in need of antimicrobial or antifungal treatment. Exemplary antibiotic and antifungal agents are described herein. The composition can be administered via any pharmaceutically acceptable method such as inhalation or orally.

In yet another embodiment, encompassed is a composition comprising a nanosized chitosan-asthma drug conjugate prepared according to the invention and methods of making and using the same. The methods of the invention comprise administering the nanosized chitosan-asthma drug conjugate to a subject in need; i.e., a subject having asthma and/or in need of asthma treatment. Exemplary asthma drugs are described herein. The composition can be administered via any pharmaceutically acceptable method such as inhalation.

The nanosized chitosan-drug conjugates preferably have an average particle size of less than about 1000 nm. In other embodiments of the invention, the chitosan-drug conjugates have an average particle size of less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm.

Another aspect of the invention is directed to nanosized chitosan-drug conjugates having mucoadhesive properties. Compositions comprising such mucoadhesive chitosan-drug conjugates can exhibit enhanced interaction with the intestinal epithelium following in vivo administration, thereby resulting in improved bioavailability and a potentially lower dosage of drug needed to obtain the desired therapeutic effect.

The compositions of the invention can be formulated into any suitable dosage form. Exemplary pharmaceutical dosage forms include, but are not limited to: (1) dosage forms for administration selected from the group consisting of oral, pulmonary (inhalation), intravenous, rectal, otic, ophthalmic, colonic, parenteral, intracisternal, intravaginal, intraperitoneal, local, buccal, nasal, and topical administration; (2) dosage forms selected from the group consisting of liquid dispersions, gels, aerosols, ointments, creams, tablets, sachets and capsules; (3) dosage forms selected from the group consisting of lyophilized formulations, fast melt formulations, controlled release formulations, delayed release formulations, extended release formulations, pulsatile release formulations, and mixed immediate release and controlled release formulations; or (4) any combination thereof.

A. Increased Solubility, Bioavailability and Lower Drug Dosages

The compositions of the invention comprising a nanosized chitosan-drug conjugate preferably exhibit increased bioavailability and less variable bioavailability at the same dose of the same drug, require smaller doses, and show longer plasma half-life as compared to non-chitosan conjugate formulations of the same drug.

In one aspect of the invention, pharmaceutical nanosized chitosan-drug conjugate compositions have enhanced bioavailability such that the drug dosage can be reduced, resulting in a decrease in toxicity associated with such drugs. It has been surprisingly found in the present invention that stable compositions of chitosan-drug conjugates can be formed that permit therapeutic levels at desirably lower dosage.

Greater bioavailability of the nanosized chitosan-drug conjugate compositions of the invention can enable a smaller solid dosage size. This is particularly significant for patient populations such as the elderly, juvenile, and infant.

In one embodiment of the invention, bioavailability of the drug present in the nanosized chitosan-drug conjugates is increased by about 10%. In other embodiments of the invention, bioavailability of the drug present in the nanosized chitosan-drug conjugates is increased by about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 20%, or about 300%. In yet other embodiments of the invention, bioavailability of the drug present in the nanosized chitosan-drug conjugates is increased by about 2 times, 3 times, 4 times, 5 times, about 6 times, about 7 times, about 8 times, about 9 times, about 10 times, about 15 times, about 20 times, about 25 times, or about 30 times. In particular, the improved bioavailability can be observed with oral dosage formulations.

In addition, the compositions of the invention comprising a nanosized chitosan-drug conjugate preferably exhibits increased water solubility, at the same dose of the same drug, as compared to non-chitosan conjugate formulations of the same drug. In one embodiment, the water solubility of the component drug is increased by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 20%, or about 300%. In yet other embodiments of the invention, water solubility of the component drug is increased by about 5 times, about 15 times, about 20 times, about 30 times, about 40 times, about 50 times, about 60 times, about 70 times, about 80 times, about 90 times, about 100 times, about 110 times, about 120 times, about 130 times, about 140 times, about 150 times, about 160 times, about 170 times, about 180 times, about 190 times, or about 200 times.

B. Improved Pharmacokinetic Profiles

The invention also preferably provides compositions comprising a nanosized chitosan-drug conjugate according to the invention having a desirable pharmacokinetic profile when administered to mammalian subjects. The desirable pharmacokinetic profile of the orally administered drug present in the nanosized chitosan complex—a statin, chemotherapeutic agent, antibiotic, antifungal or asthma drug—preferably includes, but is not limited to: (1) that the Tₘₐₓ of a statin, chemotherapeutic agent, antibiotic, antifungal or asthma drug, when assayed in the plasma of a mammalian subject following administration is preferably less than the Tₘₐₓ for a conventional, non-chitosan nanosized conjugate form of the same statin, chemotherapeutic agent, antibiotic, antifungal or asthma drug, administered at the same dosage; (2) that the Cₘₐₓ of a statin, chemotherapeutic agent, antibiotic, antifungal or asthma drug when assayed in the plasma of a mammalian subject following administration is preferably greater than the Cₘₐₓ for a conventional, non-chitosan nanosized conjugate form of the same statin, chemotherapeutic agent, antibiotic, antifungal or asthma drug, administered at the same dosage; and/or (3) that the AUC of a statin, chemotherapeutic agent, antibiotic, antifungal or asthma drug when assayed in the plasma of a mammalian subject following administration, is preferably greater than the AUC for a non-chitosan nanosized conjugate form of the same statin, chemotherapeutic agent, antibiotic, antifungal or asthma drug, administered at the same dosage.

The desirable pharmacokinetic profile, as used herein, is the pharmacokinetic profile measured after the initial dose of a statin, chemotherapeutic agent, antibiotic, antifungal or asthma drug. The nanosized chitosan-drug conjugate compositions can be formulated in any way as described herein.

A preferred nanosized chitosan-drug conjugate composition of the invention, comprising a statin, chemotherapeutic agent, antibiotic, antifungal or asthma drug, exhibits in comparative pharmacokinetic testing with a nanosized chitosan conjugate form of the same statin, chemotherapeutic agent, antibiotic, antifungal or asthma drug, administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, or not greater than about 10% of the Tₘₐₓ, exhibited by the non-chitosan-drug nanosized conjugate composition of the same statin, chemotherapeutic agent, antibiotic, antifungal or asthma drug.

A preferred nanosized chitosan-drug conjugate composition of the invention, comprising a statin, chemotherapeutic agent, antibiotic, antifungal or asthma drug, exhibits in comparative pharmacokinetic testing with a non-chitosan-drug nanosized conjugate composition of the same statin, chemotherapeutic agent, antibiotic, antifungal or asthma drug, administered at the same dosage, a Cₘₐₓ which is at least about 10%, at least about 20%, at least about 30%, at least about 40%, at least about 50%, at least about 60%, at least about 70%, at least about 80%, at least about 90%, or at least about 100% greater than the Cₘₐₓ exhibited by the non-chitosan-drug nanosized conjugate composition of the same statin, chemotherapeutic agent, antibiotic, antifungal or asthma drug.

A nanosized chitosan-drug conjugate composition of the invention, comprising a statin, chemotherapeutic agent, antibiotic, antifungal or asthma drug, exhibits in comparative pharmacokinetic testing with a non-chitosan-drug nanosized conjugate composition of the same statin, chemotherapeutic agent, antibiotic, antifungal or asthma drug, administered at the same dosage, an AUC which is at least about 10%, at least about 20%, at least about 30%, at least about 40%, at least about 50%, at least about 60%, at least about 70%, at least about 80%, at least about 90%, or at least about 100% greater than the AUC exhibited by the non-chitosan-drug nanosized conjugate composition of the same statin, chemotherapeutic agent, antibiotic, antifungal or asthma drug.

Any nanosized chitosan-drug conjugate composition giving the desired pharmacokinetic profile is suitable for administration according to the present methods. Exemplary types of formulations giving such profiles are liquid dispersions, gels, aerosols, ointments, creams, solid dose forms, etc. comprising a chitosan-drug conjugate composition according to the invention.

C. The PK Profiles of the Chitosan-Drug Conjugate Compositions are not Affected by the Fed or Fasted State of a Subject

The invention encompasses a nanosized chitosan-drug conjugate composition of the invention, comprising a statin, chemotherapeutic agent, antibiotic, antifungal or asthma drug, wherein the pharmacokinetic profile of the statin, chemotherapeutic agent, antibiotic, antifungal or asthma drug is preferably not substantially affected by the fed or fasted state of a subject ingesting the composition, when administered to a human. This means that there is no substantial difference in the quantity of drug absorbed or the rate of drug absorption when the nanosized chitosan-drug conjugate compositions are administered in the fed versus the fasted state.

The invention also encompasses a nanosized chitosan-drug conjugate composition of the invention, comprising a statin or chemotherapeutic agent, in which administration of the composition to a subject in a fasted state is bioequivalent to administration of the composition to a subject in a fed state. “Bioequivalency” is preferably established by a 90% Confidence Interval (CI) of between 0.80 and 1.25 for both Cₘₐₓ and AUC under U.S. Food and Drug Administration regulatory guidelines, or a 90% CI for AUC of between 0.80 to 1.25 and a 90% CI for Cₘₐₓ of between 0.70 to 1.43 under the European EMEA regulatory guidelines (Tₘₐₓ is not relevant for bioequivalency determinations under USFDA and EMEA regulatory guidelines).

Benefits of a dosage form which substantially eliminates the effect of food include an increase in subject convenience, thereby increasing subject compliance, as the subject does not need to ensure that they are taking a dose either with or without food. This is significant, as with poor subject compliance an increase in the medical condition for which the drug is being prescribed may be observed: e.g., poor lipid control for statins, recurrent or resistant microbial infections for antibiotics or antifungals, poor cancer treatment for chemotherapeutic agents, and asthma attacks for asthma drugs. Moreover, for patients having severe nausea, such as patients taking chemotherapeutic agents, the requirement to take medication with food to obtain optimal drug absorption can be difficult if not impossible.

The difference in absorption of the nanosized chitosan-drug conjugate composition of the invention, comprising a statin, chemotherapeutic agent, antibiotic, antifungals or asthma drug, when administered in the fed versus the fasted state, preferably is less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, or less than about 3%.

### II. Definitions

The present invention is described herein using several definitions, as set forth below and throughout the application.

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The terms “pharmaceutically acceptable” or “pharmacologically acceptable,” as used herein, refer to compositions that do not substantially produce adverse allergic or immunological reactions when administered to a host (e.g., an animal or a human). Such formulations include any pharmaceutically acceptable dosage form. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like.

The phrase “poorly water-soluble drugs” as used herein refers to drugs having a solubility in water of less than about 30 mg/mL, less than about 20 mg/mL, less than about 10 mg/mL, less than about 1 mg/mL, less than about 0.1 mg/mL, less than about 0.01 mg/mL or less than about 0.001 mg/mL.

The term “subject” as used herein refers to organisms to be treated by the compositions of the present invention. Such organisms include animals (domesticated animal species, wild animals), and humans.

### III. Nanosized Chitosan-Drug Conjugates

The present invention encompasses nanosized chitosan-drug conjugates where the drug is a statin, chemotherapeutic agent, antibiotic, antifungal or asthma drug. Any poorly water-soluble statin, chemotherapeutic agent, antibiotic, antifungal or asthma drug can be conjugated to chitosan using the conjugation method described herein.

A. Statins

As used herein “statin” means any HMG-CoA Reductase Inhibitor (including their analogs), or a salt thereof. Preferably, the statin is poorly water-soluble. Such statin compounds include, but are not limited to, atorvastatin (Lipitor®) and other 6-[2-(substituted-pyrrol-1-yl)alkyl]pyran-2-ones and derivatives as disclosed in U.S. Pat. No. 4,647,576); fluvastatin (Lescol®), lovastatin (Mevacor®, Altocor®, Altoprev®), pravastatin (Pravachol®, Selektine®, Lipostat®), pitavastatin (Livalo®, Pitava®), rosuvastatin (Crestor®), simvastatin (Zocor®, Lipex®), velostatin, fluindostatin (Sandoz XU-62-320), pyrazole analogs of mevalonolactone derivatives, as disclosed in PCT application WO 86/03488; rivastatin and other pyridyldihydroxyheptenoic acids, as disclosed in European Patent 491226A; Searle's SC-45355 (a 3-substituted pentanedioic acid derivative); dichloroacetate; imidazole analogs of mevalonolactone, as disclosed in PCT application WO 86/07054; 3-carboxy-2-hydroxy-propane-phosphonic acid derivatives, as disclosed in French Patent No. 2,596,393; 2,3-di-substituted pyrrole, furan, and thiophene derivatives, as disclosed in European Patent Application No. 0221025; naphthyl analogs of mevalonolactone, as disclosed in U.S. Pat. No. 4,686,237; octahydronaphthalenes, such as those disclosed in U.S. Pat. No. 4,499,289; keto analogs of mevinolin (lovastatin), as disclosed in European Patent Application No. 0,142,146 A2; phosphinic acid compounds; as well as other HMG CoA reductase inhibitors. In one embodiment, the statin is not atorvastatin.

Preferred statins for the compositions of the invention include atorvastatin and rosuvastatin. Atorvastatin is used for lowering blood cholesterol. It also stabilizes plaque and prevents strokes through anti-inflammatory and other mechanisms. Like all statins, atorvastatin works by inhibiting HMG-CoA reductase, an enzyme found in liver tissue that plays a key role in production of cholesterol in the body. The primary uses of atorvastatin is for the treatment of dyslipidemia and the prevention of cardiovascular disease.

Atorvastatin undergoes rapid oral absorption, with an approximate time to maximum plasma concentration (Tₘₐₓ) of 1-2 hours. The absolute bioavailability of the drug is approximately 14%; however, the systemic availability for HMG-CoA reductase activity is approximately 30%. Atorvastatin undergoes high intestinal clearance and first-pass metabolism, which is the main cause for the low systemic availability. Administration of atorvastatin with food produces a 25% reduction in Cₘₐₓ (rate of absorption) and a 9% reduction in AUC (extent of absorption), although food does not affect the plasma LDL-C-lowering efficacy of atorvastatin. Evening dose administration is known to reduce the Cₘₐₓ (rate of absorption) and AUC (extent of absorption) by 30% each. In particular, there is a need for oral dosage forms of atorvastatin having improved bioavailability and reduced food effect. The nanosized chitosan-statin compositions of the invention satisfy this need.

Rosuvastatin (marketed by AstraZeneca as Crestor®) is a member of the drug class of statins, used to treat high cholesterol and related conditions, and to prevent cardiovascular disease. Rosuvastatin has structural similarities with most other synthetic statins, e.g., atorvastatin, cerivastatin, pitavastatin, but rosuvastatin unusually also contains sulfur. Rosuvastatin is approved for the treatment of high LDL cholesterol (dyslipidemia), total cholesterol (hypercholesterolemia), and/or triglycerides (hypertriglyceridemia). In February 2010, rosuvastatin was approved by the FDA for the primary prevention of cardiovascular events.

The results of the JUPITER trial (2008) suggested rosuvastatin may decrease the relative risk of heart attack and stroke in patients without hyperlipidemia, but with elevated levels of highly sensitive C-reactive protein. This could strongly impact medical practice by placing many patients on statin prophylaxis who otherwise would have been untreated. As a result of this clinical trial, the FDA approved rosuvastatin for the primary prevention of cardiovascular events. As with all statins, there is a concern of rhabdomyolysis.

In clinical pharmacology studies in man, peak plasma concentrations of rosuvastatin were reached 3 to 5 hours following oral dosing, and rosuvastatin's approximate elimination half life is 19 h. Both Cₘₐₓ and AUC increased in approximate proportion to CRESTOR® (rosuvastatin calcium) dose. The absolute bioavailability of rosuvastatin is approximately 20%. Administration of rosuvastatin with food did not affect the AUC of rosuvastatin. The AUC of rosuvastatin does not differ following evening or morning drug administration. In particular, there is a need for oral dosage forms of rosuvastatin having improved bioavailability. The nanosized chitosan-statin compositions of the invention satisfy this need.

B. Chemotherapeutic Agents

Examples of chemotherapeutic agents include, but are not limited to, (1) taxanes, such as paclitaxel and docetaxel; (2) alkylating agents such as melphalan, chlorambucil, cyclophosphamide, mechlorethamine, uramustine, ifosfamide, carmustine, lomustine, streptozocin, busulfan, thiotepa, cisplatin, carboplatin, nedaplatin, oxaliplatin, satraplatin, triplatin, tetranitrate, procarbazine, altretamine, dacarbazine, mitozolomide, and temozolomide; (3) anti-metabolites such as azathioprine, mercaptopurine, Azathioprine, Mercaptopurine, Thioguanine Fludarabine, Pentostatin, cladribine, 5-fluorouracil (5FU), Floxuridine (FUDR), Cytosine arabinoside (Cytarabine), 6-azauracil, methotrexate, trimethoprim, pyrimethamine, pemetrexed, raltitrexed, pemetrexed, Vincristine, Vinblastine, Vinorelbine, Vindesine, Etoposide, and teniposide; (4) Topoisomerase inhibitors, such as camptothecins, irinotecan, topotecan, amsacrine, etoposide, etoposide phosphate, and teniposide; (5) Cytotoxic antibiotics, such as actinomycin, anthracyclines, doxorubicin, daunorubicin, valrubicin, idarubicin, epirubicin, bleomycin, plicamycin, and mitomycin.

Preferred chemotherapeutic agents of the invention are taxanes such as paclitaxel and docetaxel. The taxanes are a class of anticancer agents that bind to and stabilize microtubules causing cell-cycle arrest and apoptosis (cell death).

Paclitaxel is a mitotic inhibitor used in cancer chemotherapy. It is a poorly water-soluble compound. Commercially available paclitaxel formulations are dissolved in Cremophor EL and ethanol (Taxol®). Much of the clinical toxicity of paclitaxel is associated with the solvent Cremophor EL in which it is dissolved for delivery. In a newer formulation, paclitaxel is bound to albumin (Abraxane®). Paclitaxel is used to treat patients with breast, ovarian, lung, bladder, prostate, melanoma, head and neck cancer, esophageal, as well as other types of solid tumor cancers, and advanced forms of Kaposi's sarcoma. Paclitaxel is also used for the prevention of restenosis.

Because of its large molecular weight, absorption of paclitaxel from the peritoneum is reduced. Paclitaxel is also a preferred chemotherapeutic agent as it is rapidly cleared by the liver. However, local toxic effects such as abdominal pain and life-threatening hypersensitivity reactions of the current Taxol® formulation have limited the use of paclitaxel and prompted the need for newer and safer paclitaxel formulations. Markman M., Lancet Oncol., 4:277-83 (2003); Gelderblom et al., “Cremophor EL. The drawbacks and advantages of vehicle selection for drug formulation,” Eur J Cancer, 37:1590-8 (2001).

Docetaxel (as generic or under the trade name Taxotere®) is a clinically well-established anti-mitotic chemotherapy medication. It is used mainly for the treatment of breast, ovarian, prostate, and non-small cell lung cancer. Clinical data has shown docetaxel to have cytotoxic activity against breast, colorectal, lung, ovarian, prostate, liver, renal, gastric, head and neck cancers, and melanoma. Docetaxel is a white powder and is the active ingredient available in 20 mg and 80 mg Taxotere single-dose vials of concentrated anhydrous docetaxel in polysorbate 80. The solution is a clear brown-yellow containing 40 mg docetaxel and 1040 mg polysorbate 80 per mL. Taxotere® is distributed in a blister carton containing one single-dose vial of Taxotere (docetaxel) preparation in sterile pyrogen-free anhydrous polysorbate 80, and a single dose Taxotere solvent vial containing ethanol in saline to be combined and diluted in a an infusion bag containing 0.9% sodium chloride or 5% glucose for administration. The docetaxel and solvent vials are combined and the required dose is drawn from this solution.

Intravenous administration of docetaxel results in 100% bioavailability and absorption is immediate. In practice, docetaxel is administered intravenously only to increase dose precision However, oral bioavailability has been found to be 8%±6% on its own and, when co-administered with cyclosporine, bioavailability increased to 90%±44%. However, as cyclosporine is an immunosuppressant, there is a need for oral formulations of taxanes such as docetaxel having a high bioavailability.

C. Antibiotics (Antimicrobials)

Antibacterial antibiotics are commonly classified based on their mechanism of action, chemical structure, or spectrum of activity. Most target bacterial functions or growth processes. Those that target the bacterial cell wall (penicillins and cephalosporins) or the cell membrane (polymixins), or interfere with essential bacterial enzymes (rifamycins, lipiarmycins, quinolones, and sulfonamides) have bactericidal activities. Those that target protein synthesis (macrolides, lincosamides and tetracyclines) are usually bacteriostatic (with the exception of bactericidal aminoglycosides). Four new classes of antibacterial antibiotics have recently been introduced into clinical use: cyclic lipopeptides (daptomycin), glycylcyclines (tigecycline), oxazolidinones (linezolid) and lipiarmycins (fidaxomicin). Drugs currently in clinical development include ceftolozane/tazobactam (CXA-201; CXA-101/tazobactam), ceftazidime/avibactam (ceftazidime/NXL104), ceftaroline/avibactam (CPT-avibactam; ceftaroline/NXL104), imipenem/MK-7655, plazomicin (ACHN-490), eravacycline (TP-434), and brilacidin (PMX-30063).

Exemplary antibiotics that can be incorporated into the chitosan conjugate include, but are not limited to, agents or drugs that are microbicidal and/or microbiostatic (e.g., inhibiting replication of microbes (e.g., bacteria, fungi, yeast) or inhibiting synthesis of microbial components required for survival of the infecting organism), such as almecillin, amdinocillin, amikacin, amoxicillin, amphomycin, amphotericin B, ampicillin, azacitidine, azaserine, azithromycin, azlocillin, aztreonam, bacampicillin, bacitracin, benzyl penicilloyl-polylysine, bleomycin, candicidin, capreomycin, carbenicillin, cefaclor, cefadroxil, cefamandole, cefazoline, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, chlortetracycline, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), dactinomycin, dalbavancin, dalfopristin, daptomycin, daunorubicin, demeclocycline, detorubicin, dicloxacillin, dihydrostreptomycin, dirithromycin, doxorubicin, doxycycline, epirubicin, erythromycin, eveminomycin, floxacillin, fosfomycin, fusidic acid, gemifloxacin, gentamycin, gramicidin, griseofulvin, hetacillin, idarubicin, imipenem, iseganan, ivermectin, kanamycin, laspartomycin, linezolid, linocomycin, loracarbef, magainin, meclocycline, meropenem, methacycline, methicillin, mezlocillin, minocycline, mitomycin, moenomycin, moxalactam, moxifloxacin, mycophenolic acid, nafcillin, natamycin, neomycin, netilmicin, niphimycin, nitrofurantoin, novobiocin, oleandomycin, oritavancin, oxacillin, oxytetracycline, paromomycin, penicillamine, penicillin G, penicillin V, phenethicillin, piperacillin, plicamycin, polymyxin B, pristinamycin, quinupristin, rifabutin, rifampin, rifamycin, rolitetracycline, sisomicin, spectrinomycin, streptomycin, streptozocin, sulbactam, sultamicillin, tacrolimus, tazobactam, teicoplanin, telithromycin, tetracycline, ticarcillin, tigecycline, tobramycin, troleandomycin, tunicamycin, tyrthricin, vancomycin, vidarabine, viomycin, virginiamcin, and rifampin.

Exemplary antifungal agents that can be incorporated into the nanoemulsion composition include, but are not limited to, (1) azoles (imidazoles), (2) antimetabolites, (3) allylamines, (4) morpholine, (5) glucan synthesis inhibitors (chemical family: echinocandins), (6) polyenes, (7) benoxaborales, (8) other antifungal agents, and (9) new classes of antifungal agents.

Examples of azoles include, but are not limited to, Bifonazole, Clotrimazole, Econazole, Miconazole, Tioconazole, Fluconazole, Itraconazole, Ketoconazole, Pramiconazole, Ravuconazole, Posaconazole, and Voriconazole. An example of an antimetabolite includes, but is not limited to, Flucytosine. Examples of allylamines include, but are not limited to, Terbinafine, Naftidine and amorolfine. Examples of glucan synthesis inhibitors include, but are not limited to, Caspofungin, Micafungin, and Anidulafungin. Examples of polyenes include, but are not limited to, Amphotericin B, Nystatin, and pimaricin. An example of a benoxaborale is AN2690. Other examples of antifungal agents include, but are not limited to, griseofulvin and ciclopirox. Finally, examples of new classes of antifungal agents include, but are not limited to, sodarin derivatives and nikkomycins.

D. Asthma Drugs

There are two main types of asthma medications: controller medication and quick relief or rescue medications. There are several types of long-term control medications, including, but are not limited to (1) inhaled corticosteroids, such as fluticasone (Flovent Diskus), budesonide (Pulmicort), mometasone (Asmanex Twisthaler), beclomethasone (Qvar), and ciclesonide (Alvesco); (2) leukotriene modifiers, such as montelukast (Singulair), zafirlukast (Accolate), and zileuton (Zyflo); (3) long-acting beta agonists (LABAs), such as salmeterol (Serevent), and formoterol (Foradil, Perforomist); and (4) theophylline (Theo-24, Elixophyllin, others). Examples of quick-relief or rescue medications include, but are not limited to (1) albuterol (ProAir HFA, Ventolin HFA, others), (2) levalbuterol (Xopenex HFA), (3) pirbuterol (Maxair), (4) ipratropium (Atrovent), and (5) oral corticosteroids, such as prednisone and methylprednisolone.

### IV. Fenofibrate Nanoemulsions

As noted above, in one embodiment of the invention, encompassed is a composition comprising a nanosized chitosan-statin conjugate prepared according to the invention combined with a fenofibrate nanoemulsion and methods of making and using the same. Methods of making the fenofibrate nanoemulsion are described, for example, in US 2007/0264349.

The fenofibrate nanoemulsion can be combined with a nanosized chitosan-statin conjugate according to the invention, the fenofibrate nanoemulsion can be co-administered with the chitosan-statin conjugate. “Coadministration” includes administering the fenofibrate nanoemulsion before, during, or after administration of the chitosan-statin conjugate.

The fenofibrate nanoemulsion comprises (1) a micelle component, (2) a hydro-alcoholic component, e.g., a mixture of water and water-miscible solvent, (3) an oil-in-water emulsion droplet component, and (4) a solid particle component. The fenofibrate may be in solution, as denoted in components 1 to 3, or it may be in precipitated suspension form, as is the case in component 4. In another embodiment, the fenofibrate nanoemulsion comprises globules of oil comprising dissolved fenofibrate. The globules can have a diameter of less than about 2 microns. In other embodiments of the invention, the oil globules can have a smaller diameter.

The fenofibrate nanoemulsion can be formed using classic emulsion forming techniques. See e.g., U.S. 2004/0043041. See also the method of manufacturing nanoemulsions described in U.S. Pat. Nos. 6,559,189, 6,506,803, 6,635,676, 6,015,832, and U.S. Patent Publication Nos. 20040043041, 20050208083, 20060251684, and 20070036831, and WO 05/030172, all of which are specifically incorporated by reference.

Two specific methods of making the fenofibrate nanoemulsion are described. In the first method (“Route I”), fenofibrate is milled in an emulsion base. This method requires that fenofibrate is poorly soluble or insoluble in all phases of the oil phase/lipophilic phase and the water or buffer. In the second method (“Route II”), simultaneous milling and precipitation of the fenofibrate in an emulsion base is observed. The second method requires that fenofibrate is soluble or partially soluble in one or more phases of the emulsion base; e.g., that the fenofibrate is soluble in an oil, solvent, or water or buffer.

For Route I, fenofibrate is first suspended in a mixture of a non-miscible liquid, which can comprise at least one oil, at least one solvent, and at least one buffer or water to form an emulsion base, followed by homogenization or vigorous stirring of the emulsion base. Fenofibrate nanoparticles can be produced with reciprocating syringe instrumentation, continuous flow instrumentation, or high speed mixing equipment. High velocity homogenization or vigorous stirring, producing forces of high shear and cavitation, are preferred. High shear processes are preferred as low shear processes can result in larger fenofibrate particle sizes. The resultant composition is a composite mixture of fenofibrate suspended in the emulsion droplet (nanoemulsion fenofibrate fraction) and sterically stabilized micro-/nano-crystalline fenofibrate in the media. This tri-phasic system comprises particulate fenofibrate, oil, and water or buffer. The resultant micro/nano-particulate fenofibrate has a mean particle size of less than about 3 microns. Smaller particulate fenofibrate can also be obtained, as described below.

Route II is utilized for an API that is soluble in at least one part of the emulsion base, such as the solvent. For Route II, fenofibrate is dissolved in a mixture of oil, solvent, and stabilizer to form an emulsion pre-mix. Fenofibrate remains in soluble form if water or buffer is not added to the mixture. Upon the addition of water or buffer and the application of shear forces, fenofibrate is precipitated into micro/nano-particles having a mean particle size of less than about 3 microns. Nanoparticles can be produced with reciprocating syringe instrumentation, continuous flow instrumentation, or high speed mixing equipment. High energy input, through high velocity homogenization or vigorous stirring, is a preferred process. The high energy processes reduce the size of the emulsion droplets, thereby exposing a large surface area to the surrounding aqueous environment. High shear processes are preferred, as low shear processes can result in larger particle sizes. This is followed by precipitation of nanoparticulate fenofibrate previously embedded in the emulsion base. The end product comprises fenofibrate in solution and particulate suspension, both distributed between the solvent, oil, and water or buffer. Nanoparticulate fenofibrate has at least one surface stabilizer associated with the surface thereof.

Fenofibrate is an example of an API that is poorly soluble in water but soluble in another liquid, as fenofibrate is freely soluble in 1-methyl-2-pyrrolidone or N-methyl-pyrrolidinone [NMP], slightly soluble in oil and stabilizer, while insoluble in water.

Larger oil droplets and/or fenofibrate particles can be created by simply increasing the water content, decreasing the oil-stabilizer-solvent content, or reducing the shear in forming the oil droplets.

For the emulsion base used in Route I or Route II, the preferred ratio of oil:stabilizer:solvent is about 23:about 5:about 4, respectively, on a weight-to-weight basis. The preferred ratio of the oil comprising phase to water or buffer is about 2:about 1, respectively. The oil ratio may be about 10 to about 30 parts; the solvent ratio may be about 0.5 to about 10 parts; the stabilizer ratio may be about 1 to about 8 parts, and the water may be about 20 to about 80% (w/w).

For the emulsion base used in Route I or Route II, the preferred ratio of oil:stabilizer:solvent is about 23:about 5:about 4, respectively, on a weight-to-weight basis. The preferred ratio of the oil comprising phase to water or buffer is about 2:about 1, respectively. The oil ratio may be about 10 to about 30 parts; the solvent ratio may be about 0.5 to about 10 parts; the stabilizer ratio may be about 1 to about 8 parts, and the water may be about 20 to about 80% (w/w).

In general, the emulsion globules comprising solubilized fenofibrate, fenofibrate particles, or a combination thereof have a diameter of less than about 10 microns. In other embodiments of the invention, the emulsion globules comprising solubilized fenofibrate, fenofibrate particles, or a combination thereof can have a diameter of less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm. In other embodiments of the invention, at least about 50%, at least about 60%, at least about 70%, at least about 80%, at least about 90%, at least about 95%, or at least about 99% of the emulsion globules comprising solubilized fenofibrate, fenofibrate particles, or a combination thereof can have a diameter less than the size listed above, e.g., less than about 10 microns, less than about 9 microns, etc.

In a preferred embodiment, the oil globules have a diameter of less than about 2 microns, with a mean diameter of about 1 micron preferred. In another embodiment of the invention, the oil globules are filterable through a 0.2 micron filter, such as is typically used for microbiological purification.

The range of fenofibrate concentration in the globules can be from about 1% to about 50%. The emulsion globules can be stored at between about −20 and about 40° C.

Ingredients of the fenofibrate nanoemulsion are described below. The fenofibrate nanoemulsions can be spray dried or lyophilized and formulated into any desirable pharmaceutical dosage form. The relative amounts of the fenofibrate, at least one solvent, at least one oil, at least one surfactant/detergent, and aqueous phase can vary widely. The optimal amount of the individual components depends, for example, upon one or more of the physical and chemical attributes of the surfactant selected, such as the hydrophilic lipophilic balance (HLB), melting point, and the surface tension of water solutions of the surfactant, etc.

In a first embodiment, the concentration of fenofibrate in the fenofibrate nanoemulsion can vary from about 0.05% to about 50 (w/w %). Higher concentrations of the active ingredient are generally preferred from a dose and cost efficiency standpoint. The concentration of the oil in the fenofibrate nanoemulsion can vary from about 10% to about 80% (w/w %). The concentration of the solvent in the fenofibrate nanoemulsion can vary from about 1% to about 50% (w/w %). The concentration of the at least one surfactant in the fenofibrate nanoemulsion can vary from about 0.05% to about 40% (w/w %). The amount of water can vary from about 5% to 80%. In a second embodiment, the concentration of fenofibrate in the fenofibrate nanoemulsion can vary from about 4% to about 20% (w/w %). The concentration of the oil in the fenofibrate nanoemulsion can vary from about 30% to about 50% (w/w %). The concentration of the solvent in the fenofibrate nanoemulsion can vary from about 10% to about 20% (w/w %). The concentration of the at least one surfactant in the fenofibrate nanoemulsion can vary from about 5% to about 10% (w/w %). Finally, the amount of water can vary from about 20% to 40% (w/w %).

A. Aqueous Phase

The aqueous solution is preferably a physiologically compatible solution such as water or phosphate buffered saline. The aqueous phase can comprise any type of aqueous phase including, but not limited to, water (e.g., H₂O, distilled water, tap water) and solutions (e.g., phosphate-buffered saline (PBS) solution). The water can be deionized (hereinafter “DiH₂O”). The aqueous phase may further be sterile and pyrogen free.

B. Solvents

Any suitable solvent can be used in the fenofibrate nanoemulsion, and more than one solvent can be used in the fenofibrate nanoemulsion. Exemplary solvents include, but are not limited, to alcohols, such as a C₁₋₁₂ alcohol, isopropyl myristate, triacetin, N-methyl pyrrolidinone, long-chain alcohols, polyethylene glycols, propylene glycol, and long- and short-chain alcohols, such as ethanol, and methanol. Other short chain alcohols and/or amides may be used. Mixtures of solvents can also be used in the compositions and methods of the invention.

C. Oil Phase

The oil in the fenofibrate nanoemulsion can be any cosmetically or pharmaceutically acceptable oil, and more than one oil can be used in the fenofibrate nanoemulsion. The oil can be volatile or non-volatile, and may be chosen from animal oil, vegetable oil, natural oil, synthetic oil, hydrocarbon oils, silicone oils, semi-synthetic derivatives thereof, and combinations thereof.

Exemplary oils that can be used include, for example, vegetable oils, nut oils, fish oils, lard oil, mineral oils, squalane, tricaprylin, and mixtures thereof. Specific examples of oils that may be used include, but are not limited to, almond oil (sweet), apricot seed oil, borage oil, canola oil, coconut oil, corn oil, cotton seed oil, fish oil, jojoba bean oil, lard oil, linseed oil (boiled), Macadamia nut oil, medium chain triglycerides, mineral oil, olive oil, peanut oil, safflower oil, sesame oil, soybean oil, squalene, sunflower seed oil, tricaprylin (1,2,3-trioctanoyl glycerol), wheat germ oil, and mixtures thereof.

D. Stabilizers or Surfactants

The stabilizer used in the fenofibrate nanoemulsion associates with, or adsorbs, to the surface of the particulate fenofibrate, but does not covalently bind to the fenofibrate particles. In addition, the individual stabilizer molecules are preferably free of cross-linkages. The stabilizer is preferably soluble in water. One or more stabilizers may be used in the fenofibrate nanoemulsions. As used herein, the terms “stabilizer”, “surface stabilizer”, and “surfactant” are used interchangeably.

Any suitable nonionic or ionic surfactant may be utilized in the compositions of the invention, including anionic, cationic, and zwitterionic surfactants. Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference. Exemplary stabilizers or surfactants that may be used in both Routes I and II include, but are not limited to, non-phospholipid surfactants, such as the Tween (polyoxyethylene derivatives of sorbitan fatty acid esters) family of surfactants (e.g., Tween 20, Tween 60, and Tween 80), nonphenol polyethylene glycol ethers, sorbitan esters (such as Span and Arlacel), glycerol esters (such as glycerin monostearate), polyethylene glycol esters (such as polyethylene glycol stearate), block polymers (such as Pluronics), acrylic polymers (such as Pemulen), ethoxylated fatty esters (such as Cremophore RH-40), ethoxylated alcohols (such as Brij), ethoxylated fatty acids, monoglycerides, silicon based surfactants, polysorbates, tergitol NP-40 (Poly(oxy-1,2-ethanediyl), α-(4-nonylphenol)-.omega.-hydroxy, branched [molecular weight average 1980]), and Tergitol NP-70 (a mixed surfactant—AQ=70%).

### V. Additional Ingredients

Pharmaceutical compositions according to the invention may also comprise one or more preservatives, pH adjuster, emulsifying agents, binding agents, filling agents, lubricating agents, suspending agents, sweeteners, flavoring agents, preservatives, buffers, wetting agents, disintegrants, effervescent agents, and other excipients depending upon the route of administration and the dosage form desired. Such excipients are known in the art.

Suitable preservatives in the compositions of the invention include, but are not limited to, quarternary compounds such as cetylpyridinium chloride and benzalkonium chloride, benzyl alcohol, chlorhexidine, imidazolidinyl urea, phenol, potassium sorbate, benzoic acid and its salts, bronopol, chlorocresol, paraben esters, methylparaben, propylparaben, other esters of parahydroxybenzoic acid such as butylparaben, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, alcohols such as ethyl or benzyl alcohol, phenolic compounds such as phenol, and combinations thereof. The composition can comprise a buffering agent, such as a pharmaceutically acceptable buffering agent.

The composition may further comprise at least one pH adjuster. Suitable pH adjusters in the nanoemulsion of the invention include, but are not limited to, diethyanolamine, lactic acid, monoethanolamine, triethylanolamine, sodium hydroxide, sodium phosphate, semi-synthetic derivatives thereof, and combinations thereof.

The composition can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present invention feature nanoemulsion compositions that may readily be diluted with water to a desired concentration without impairing their anti-fungal, antibacterial, or antiprotozoan properties.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet (trademark of MAFCO), bubble gum flavor, and fruit flavors, and the like.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

### VI. Pharmaceutical Compositions

The compositions of the invention may be formulated into pharmaceutical compositions that comprise the composition in a therapeutically effective amount and suitable, pharmaceutically-acceptable excipients for any pharmaceutically acceptable method of administration to a human subject in need thereof. Such excipients are well known in the art. Exemplary methods of administration include but are not limited to oral, injectable, nasal, pulmonary, and inhalation.

By the phrase “therapeutically effective amount” it is meant any amount of the composition that is effective in preventing and/or treating (1) high cholesterol or a related condition, such as heart disease; or (2) cancer or other disease where a chemotherapeutic is indicated.

The pharmaceutical compositions may be formulated for immediate release, sustained release, controlled release, delayed release, or any combinations thereof.

The compositions of the invention can be formulated into any suitable dosage form, such as liquid dispersions, oral suspensions, gels, aerosols, ointments, creams, tablets, capsules, dry powders, multiparticulates, sprinkles, sachets, lozenges, and syrups. Moreover, the dosage forms of the invention may be solid dosage forms, liquid dosage forms, semi-liquid dosage forms, immediate release formulations, modified release formulations, controlled release formulations, fast melt formulations, lyophilized formulations, delayed release formulations, extended release formulations, pulsatile release formulations, and mixed immediate release and controlled release formulations, or any combination thereof.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The compositions of the invention may also comprise adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the compositions of the invention may be is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the fibrate, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like. Besides such inert diluents, the composition can also include adjuvants, such as wetting agents, emulsifying and suspending agents, sweetening, flavoring, and perfuming agents.

One of ordinary skill will appreciate that effective amounts of a statin, chemotherapeutic agent, and fenofibrate can be determined empirically and can be employed in pure form or, where such forms exist, in pharmaceutically acceptable salt, ester, or prodrug form. Actual dosage levels of a statin, chemotherapeutic agent, and fenofibrate in the compositions of the invention may be varied to obtain an amount of the statin, chemotherapeutic agent, or fenofibrate that is effective to obtain a desired therapeutic response for a particular composition and method of administration. The selected dosage level therefore depends upon the desired therapeutic effect, the route of administration, the potency of the administered drug, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The compositions of the invention can be provided in many different types of containers and delivery systems. For example, in some embodiments of the invention, the nanoemulsion compositions are provided in a cream or other solid or semi-solid form. The nanoemulsion compositions of the invention may be incorporated into hydrogel formulations.

The compositions can be delivered (e.g., to a subject or customers) in any suitable container. Suitable containers can be used that provide one or more single use or multi-use dosages of the nanoemulsion compositions for the desired application. In some embodiments of the invention, the nanoemulsion compositions are provided in a suspension or liquid form. Such nanoemulsion compositions can be delivered in any suitable container.

### VII. Methods of the Invention

The chitosan-drug conjugate compositions of the invention can be administered to a subject via any conventional means including, but not limited to, orally, rectally, ocularly, parenterally (e.g., intravenous, intramuscular, or subcutaneous), intranasal, intracisternally, colonic, pulmonary, intravaginally, intraperitoneally, transdermally, locally (e.g., powders, ointments or drops), topically, or as a buccal or nasal spray. As used herein, the term “subject” is used to mean an animal, preferably a mammal, including a human or non-human. The terms patient and subject may be used interchangeably.

The statin compositions of the invention are useful, for example, in treating conditions such as dyslipidemia, hyperlipidemia, hypercholesterolemia, cardiovascular disorders, hypertriglyceridemia, coronary heart disease, and peripheral vascular disease (including symptomatic carotid artery disease), or related conditions; (2) as adjunctive therapy to diet for the reduction of LDL-C, total-C, triglycerides, and/or Apo B in adult patients with primary hypercholesterolemia or mixed dyslipidemia (Fredrickson Types IIa and IIb); (3) as adjunctive therapy to diet for treatment of adult patients with hypertriglyceridemia (Fredrickson Types IV and V hyperlipidemia); (4) in treating pancreatitis; (5) in treating restenosis; and/or (6) in treating Alzheimer's disease.

In one aspect, the statin compositions of the invention are useful in treating conditions that may be directly or indirectly associated with elevated and/or uncontrolled cholesterol metabolism.

In another aspect, the statin compositions of the invention, useful in treating, preventing, or lowering the risk of a cancer. The cancer can be any cancer described herein. Exemplary cancers for which statins may result in a lower cancer risk include, but are not limited to, cancers associated with a solid tumor, lymphomas, prostate, colorectal, bowel, breast and skin cancers. By exploring the effects of statins on the process of cancer at the molecular level, researchers have found that statins work against critical cellular functions that may help control tumor initiation, tumor growth, and metastasis. Specifically, statins reduce (or block) the activity of the enzyme HMG-CoA reductase and thereby reduce the levels of mevalonate and its associated products. The mevalonate pathway plays a role in cell membrane integrity, cell signaling, protein synthesis, and cell cycle progression, all of which are potential areas of intervention to arrest the cancer process. See http://www.cancer.gov/cancertopics/factsheet/prevention/statins (downloaded on Aug. 13, 2012).

The chemotherapeutic agent compositions of the invention are useful, for example, in treating a cancer and can afford efficient treatment of cancers with minimum adverse effects. Cancer, known medically as a malignant neoplasm, is a broad group of various diseases, all involving unregulated cell growth. In cancer, cells divide and grow uncontrollably, forming malignant tumors, and invade nearby parts of the body. The cancer may also spread to more distant parts of the body through the lymphatic system or bloodstream. In 2007, cancer caused about 13% of all human deaths worldwide (7.9 million). Rates are rising as more people live to an old age and as mass lifestyle changes occur in the developing world.

The cancer can be of any tissue, and includes solid tumors (generally refers to the presence of cancer of body tissues other than blood, bone marrow, or the lymphatic system) as well as hematopoietic disorders (cancers) (generally refers to the presence of cancerous cells originated from hematopoietic system). Such cancers include but are not limited to genital cancers such as testicular, ovarian, bladder, colorectal, breast, vulvar, uterine, lung (including but not limited to non-small cell lung cancer), prostate, liver, renal, gastric, melanoma, head and neck cancers, esophageal, as well as other types of solid tumor cancers, and advanced forms of Kaposi's sarcoma. Paclitaxel is also used for the prevention of restenosis.

Hematopoietic malignancies include, for example, acute lymphoblastic (lymphocytic) leukemia, chronic lymphocytic leukemia, acute myelogenous leukemia, chronic myelogenous leukemia, acute malignant myelosclerosis, multiple myeloma, polycythemia vera agnogenic myelometaplasia, Waldenstrom's macroglobulinemia, Hodgkin's lymphoma, and non-Hodgkin's lymphoma III. Solid tumors include, for example, malignant melanoma, non-small cell lung cancer, carcinoma of the stomach, ovarian carcinoma, breast carcinoma, small cell lung carcinoma, retinoblastoma, testicular carcinoma, glioblastoma, rhabdomyosarcoma, neuroblastoma, and Ewing's sarcoma.

The chitosan-antibiotic conjugate compositions can be used to treat and/or prevent any microbial infection. Additionally, the chitosan-antifungal conjugate compositions can be use to treat and/or prevent any fungal infection.

The chitosan-asthma drug conjugate compositions can be used to treat asthma or related symptoms and/or prevent asthma symptoms (e.g., shortness of breath).

### VIII. Examples

The invention is further described by reference to the following examples, which are provided for illustration only. The invention is not limited to the examples, but rather includes all variations that are evident from the teachings provided herein. All publicly available documents referenced herein, including but not limited to U.S. patents, are specifically incorporated by reference.

### Example 1

### Preparation of Atorvastatin-Chitosan Conjugate

The purpose of this example was to describe preparation of an atorvastatin-chitosan conjugate.

Materials: AT (Form I) was obtained as a gift sample from Lupin Ltd. (Pune, India). Chitosan (CH) (ChitoClear™, degree of deacetylation 96%; viscosity 15 cp) was obtained from Primex ehf (Siglufjordur, Iceland). 1-Ethyl-3-(3-dimethyl aminopropyl) carbodiimide (EDC) was purchased from Himedia Laboratories (Mumbai, India). All other chemicals were of analytical grade and were used as received from Merck Ltd. (Mumbai, India).

Synthesis and characterization of CH-AT conjugate: As shown in FIG. 1, CH-AT conjugate was prepared by using amide coupling reaction. A 10% (w/v) solution of AT in methanol (5 mL) was activated by EDC (125 mM, 1 mL) treatment for 4 h at room temperature to afford an ester form of AT. Separately, 1% (w/v) aqueous CH solution was prepared after hydrating CH with 1 N HCl (5 mL). The methanolic solution of AT was then added dropwise to the aqueous acidic CH solution under continuous magnetic stirring. Throughout the experiment, pH was maintained in the range of 5-6. After stirring for 24 h at room temperature, the excess reagent and the corresponding acylisourea (by-product after coupling) was removed by washing with distilled water. The reaction mixture was then purified using ultrafiltration, after which the CH-AT conjugate was lyophilized. The conjugate was then characterized by using ¹H NMR (300 MHz, Bruker Biospin, Germany) and FT-IR spectrometry (Tensor 27, Bruker Biospin, Germany) and quantified by ultra high-pressure liquid chromatography (UHPLC) (Waters Acquity™, MA, USA).

Preparation of CH-AT nano-conjugate: Nano-sizing of CH-AT conjugate was achieved using HPH technique. Briefly, 100 mg of the synthesized conjugate was dispersed in deionized water at a concentration of 0.1% (w/v). The suspension thus formed was allowed to pass through a high-pressure homogenizer (Nano DeBEE, BEE International Inc., MA, USA) to obtain nano-conjugates. CH-AT nano-conjugates were collected by lyophilization.

### Example 2

### Nano-Sizing of Atorvastatin-Chitosan Conjugate

The purpose of this example was to describe nano-sizing of an atorvastatin-chitosan conjugate.

Nano-sizing of CH-AT conjugate was achieved using high pressure homogenization (HPH) techniques. Briefly, 100 mg of the synthesized conjugate was dispersed in deionized water at a concentration of 0.1% (w/v). The suspension thus formed was allowed to pass through a high-pressure homogenizer (Nano DeBEE, BEE International Inc., MA, USA) to obtain nano-conjugates. CH-AT nano-conjugates were collected by lyophilization.

Results: To further enhance the solubility, CH-AT nano-conjugates were prepared by homogenizing CH-AT conjugates using HPH. Nanonization using wet-milling by HPH was selected since the thermal energy generated during wet-milling is lower than that generated by dry-mills as the drug is suspended in aqueous media. The influence of operating pressure and the number of homogenization cycles on the conjugate particle size was studied. The results of the particle size measurements obtained after homogenization are presented in Table 1.

TABLE 1
Effect of high pressure homogenization
process variables on particle size.

| Pressure (psi) | No. of Cycles | Particle size (nm) |
|---|---|---|
| 0 | 0 | 58.9 ± 11.8 μm |
| 20,000 | 1 | 978.6 ± 18.9* |
| | 2 | 710.0 ± 20.5* |
| | 3 | 693.7 ± 19.2* |
| 30,000 | 1 | 770.7 ± 16.9* |
| | 2 | 645.2 ± 20.1* |
| | 3 | 648.5 ± 12.4* |
| 40,000 | 1 | 392.8 ± 13.8* |
| | 2 | 215.3 ± 14.2* |
| | 3 | 214.8 ± 15.8* |

Data are shown as the means ± SD, (n = 3).
*p < 0.001 compare to non-homogenized sample.

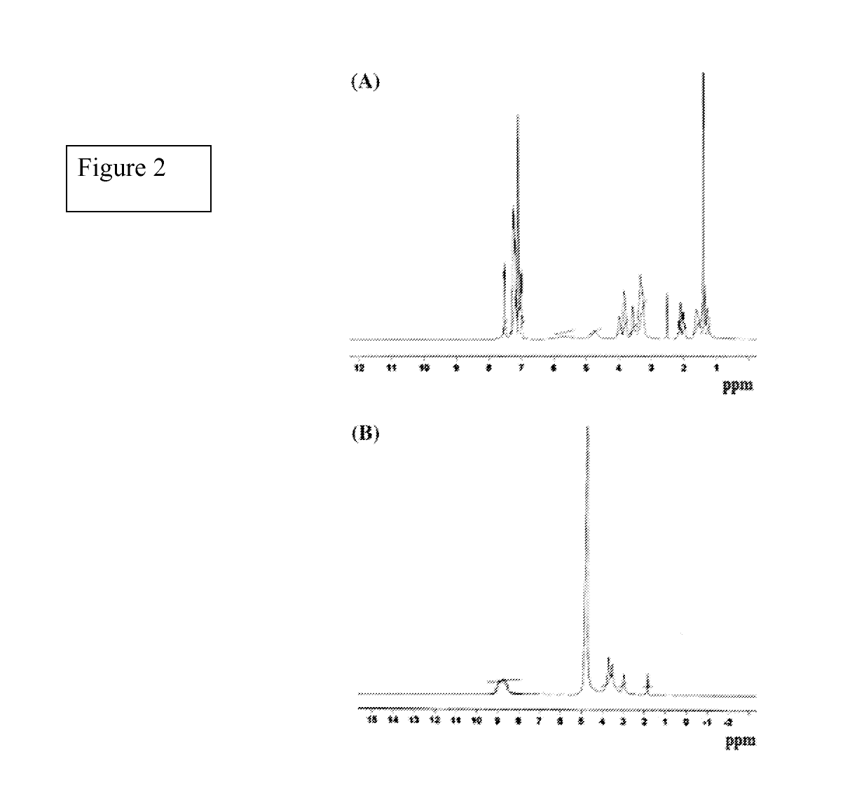

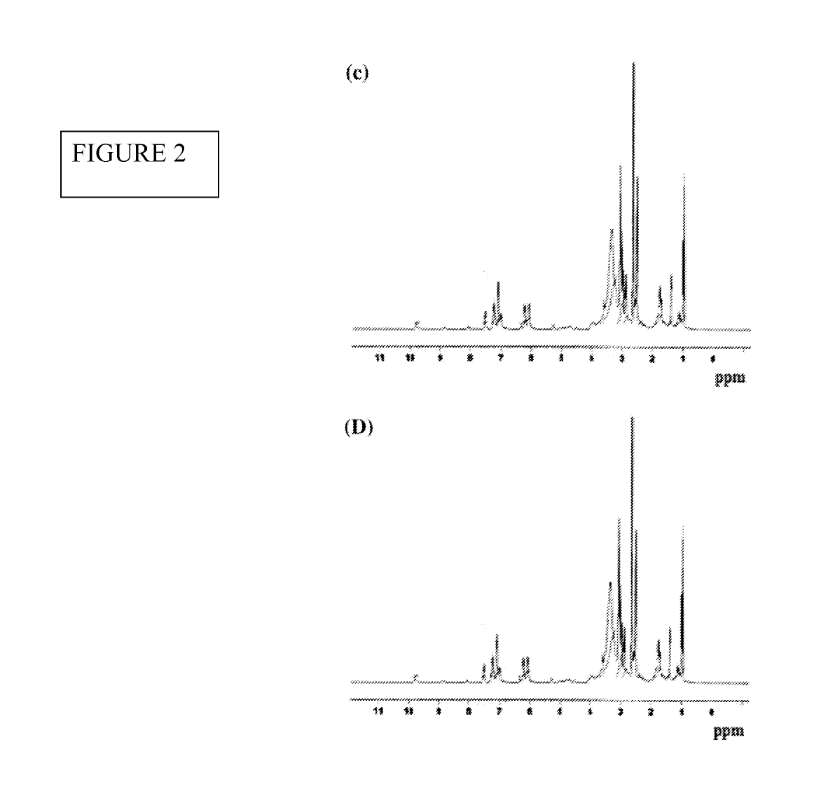

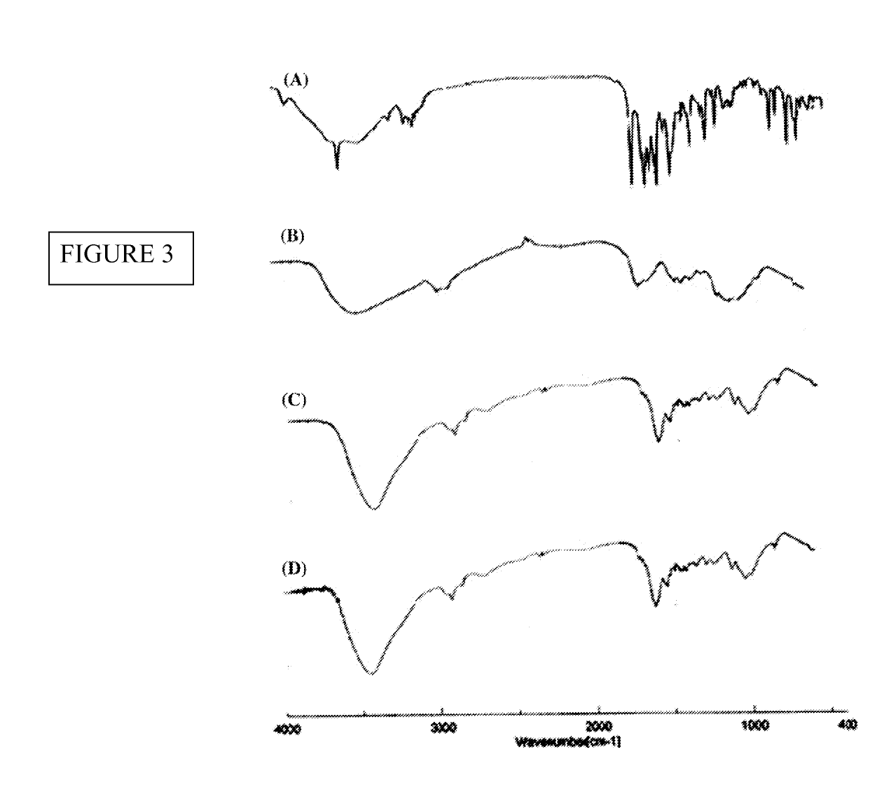

A significant reduction in particle size of CH-AT conjugate was observed after homogenization. The data also showed that the particle size had an inverse relationship with both homogenization pressure and number of cycles, and obviously mean particle size decreased with an increase in pressure or number of homogenization cycles. Conjugates with the lowest particle size were obtained after 2 homogenization cycles at 40,000 psi pressure. Increasing the number of homogenization cycles from 2 to 3 did not result in a further decrease in particle size indicating the attainment of saturation levels. Absence of any chemical change during nanonization was confirmed by ¹H NMR and FT-IR analysis. The ¹H NMR (FIG. 2D) and FT-IR spectra (FIG. 3D) of CH-AT nano-conjugate were superimposable to the corresponding spectra of CH-AT conjugate, indicating that no chemical change occurred during nanonization.

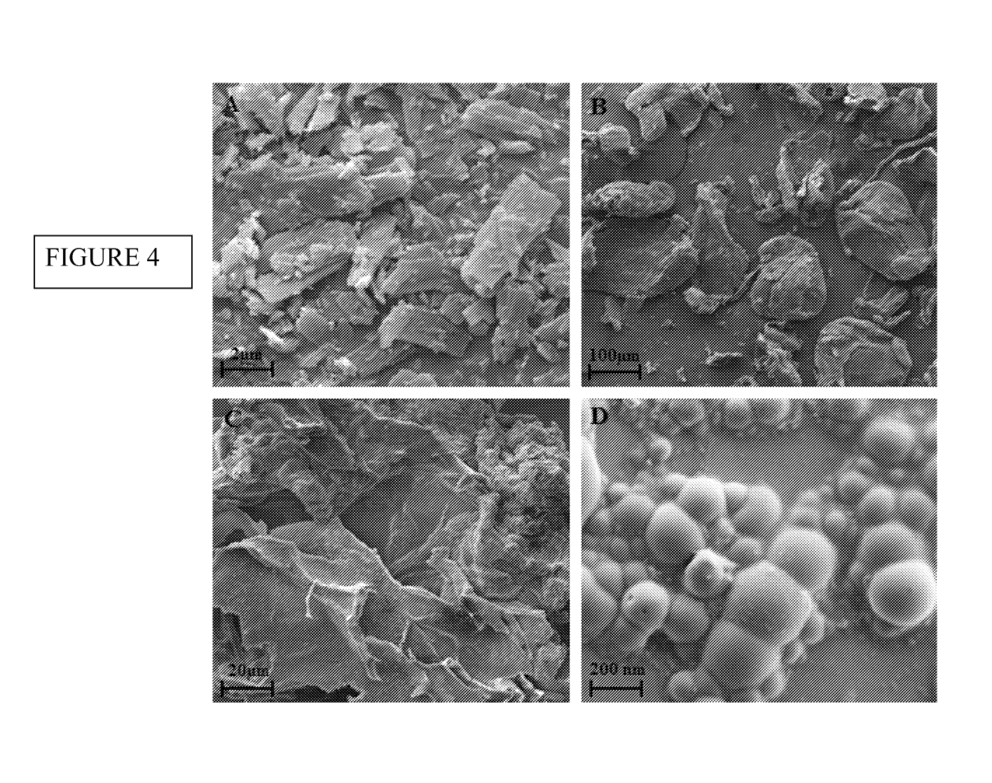

Using SEM, the homogeneity and effectiveness of the HPH milling process was readily evident. SEM micrographs of AT, CH, CH-AT conjugate and CH-AT nano-conjugate are shown in FIG. 4. In addition to particle size analysis, the SEM micrographs further provided the evidence that wet milling resulted in significant reduction of particle size. The SEM micrographs of pure AT revealed large crystalline blocks with rough surface (FIG. 4A). The surface of the CH was uniform with appearance of flaws (FIG. 4B). The formation of CH-AT conjugate resulted in a scaffold-like structure (FIG. 4C), while on homogenization, the formation of CH-AT nano-conjugate presents a smooth surface morphology of nanoconjugates (FIG. 4D).

### Example 3

### Characterizing the Nano-Sized Atorvastatin-Chitosan Conjugate

The purpose of this example was to describe characterization of the nano-sized atorvastatin-chitosan conjugates prepared in Example 2.

Nano-conjugate morphology: Morphological characteristics of the nano-conjugates were observed by scanning electron microscope (SEM, EVO LS 10, Zeiss, Carl Zeiss Inc., Germany) operating at an accelerating voltage of 13.52 kV under high vacuum. Freshly prepared nano-conjugate sample was fixed to aluminum stubs with double-sided carbon adhesive tape, sputter-coated with conductive gold-palladium and observed using SEM.

Nano-conjugate size and zeta potential: Measurement of particle size, zeta potential and polydispersity of nano-conjugates was done using Zetasizer (Nano ZS, Malvern Instruments, Malvern, UK), which is based on the principle of dynamic light scattering (DLS). All DLS measurements were done in triplicate at 25° C. at a detection angle of 90°. For zeta potential measurements, disposable capillary cell with a capacity of 1 mL was used. To obtain complete dispersion, the nano-conjugates were dispersed in Marcol 52 (Exxon Mobil Co., USA) and sonicated for 10 min at 120 W power (Branson 8210, Branson Ultrasonics Co., Danbury, Conn., USA).

Nano-conjugate crystallinity: The physical form of the lyophilized nano-conjugates was determined by powder X-ray diffraction (XRD, X'pert pro, Pan Analytical, Netherland) over a range of 20 from 5° to 60° with Ni-filtered Cu-Kαa radiation. The scan speed was 3 min⁻¹.

Solubility studies: To evaluate solubility, excess of AT, CH-AT conjugate and CH-AT nano-conjugate were added to the deionized water (10 mL) in screw-capped tubes placed in a water jacketed vessel linked to a temperature-controlled water bath maintained at 37±0.1° C. for 48 h. Continuous agitation was provided by overhead stirring. Each sample was centrifuged (REMI, Mumbai, India) at 18,000 rpm for 30 min and the respective clear supernatants containing released drug were diluted with methanol and analyzed by UHPLC as described below.

Acidic degradation studies: Stability of the drug and the formulation in conditions simulating the gastric environment was determined by adding 10 lg of AT and CH-AT nano-conjugate to 10 mL of 1 N HCl and mixture was refluxed at 80° C. At designated time points, 3 mL of the sample was withdrawn and assayed for drug concentration by UHPLC method as described below.

Measurements of mucoadhesiveness using small intestinal surfaces: The mucoadhesive property of the suspension of AT, CH-AT conjugate and CH-AT nano-conjugate were evaluated by an in vitro adhesion testing method known as the wash-off method. Freshly-excised pieces of intestinal mucosa from rat were mounted onto glass slides (3×1 sq. in.) with cyanoacrylate glue. Two glass slides were connected with a suitable support. About 50 lL of each sample was spread onto each wet rinsed tissue specimen, and immediately thereafter, the support was hung onto the arm of a USP tablet disintegrating test machine. When the disintegrating test machine was operated, the tissue specimen was given a slow, regular up-and-down movement in the test fluid (400 mL) at 37° C. contained in a 1000 mL vessel of the machine. At the end of 4 h, the machine was stopped and the remaining amount of drug adhering to the tissue was quantified by the UHPLC method (described below).

UHPLC analysis: AT was analyzed by UHPLC with a Waters Acquity™ UPLC system (Serial No #F09 UPB 920 M; Model Code #UPB; Waters, MA, USA). Chromatographic separation was performed on an Acquity UPLC BEH C18 (100 mm×2.1 mm, 1.7 lm) column. The mobile phase was composed of 0.05 M NaH₂PO₄ buffer:methanol (30:70 (v/v)), adjusted to a pH of 5.1 and a flow rate of 1.0 mL/min. The detection wavelength was set at 247 nm and the retention time was 3.9 min. For the analysis of the samples from receptor solution, aliquots of 20 lL from each sample were injected via the manual injector into a HPLC system. Plasma samples were first extracted with ethyl acetate, vortexed and centrifuged at 10,000 rpm for 15 min. The supernatant was evaporated to dryness and the residue was reconstituted with the mobile phase. All the samples were filtered through a 0.11 μm pore size membrane filter before injection. The assay was linear (r²=0.9995) in the concentration range of 0.01-50 μg/mL with a detection limit of 0.005 μg/mL. The percentage recovery ranged from 98.0% to 101.2%. No interference from the formulation components was observed.

Results: The conjugate was characterized by ¹H NMR, showing peaks corresponding to both CH and AT, and a distinctive peak at value of 9.89 owing to amide bond formation (FIG. 2C). The same has been confirmed by a distinctive peak at 1700 cm⁻¹ in FT-IR spectrum of CH-AT conjugate (FIG. 3C). Further, the absence of unsaturated carbon-carbon double bond peaks at 1420 cm⁻¹ (FIG. 3B) and displacement of the secondary amine deformation band from 1550 (FIG. 3A) to 1480 cm⁻¹ (FIG. 3C), suggests that the coupling reaction had occurred between the amino group of chitosan and the carboxylic group of AT. The weight percentage (% w/w) of AT in the CH-AT conjugate as quantified using the UHPLC method was ˜15% w/w).

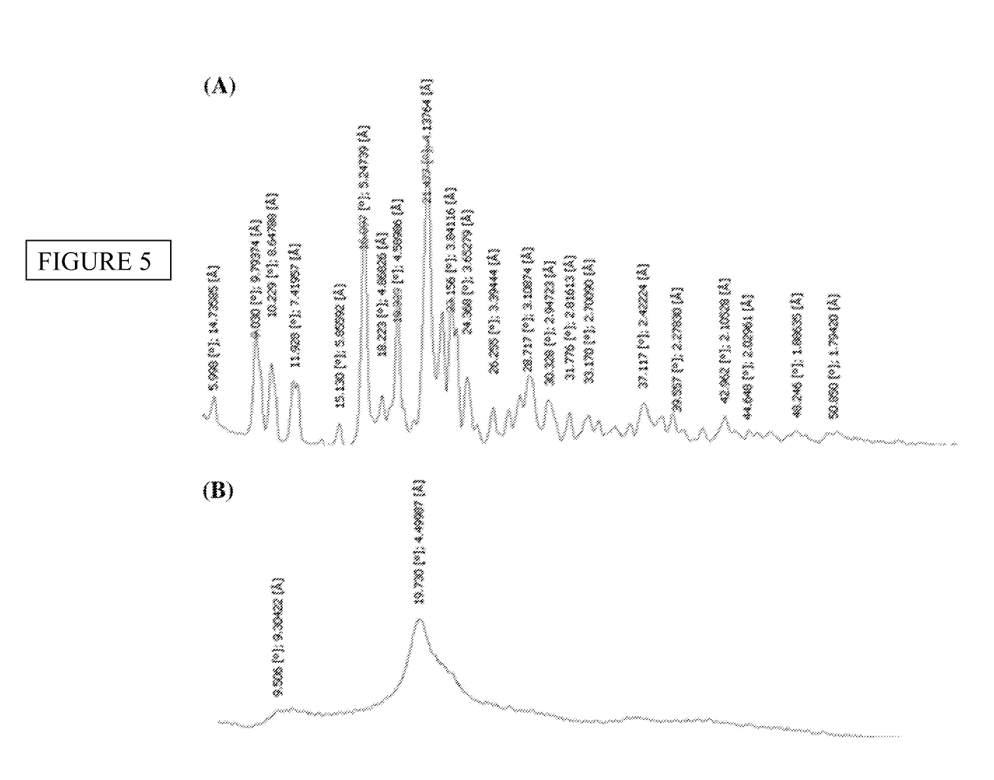

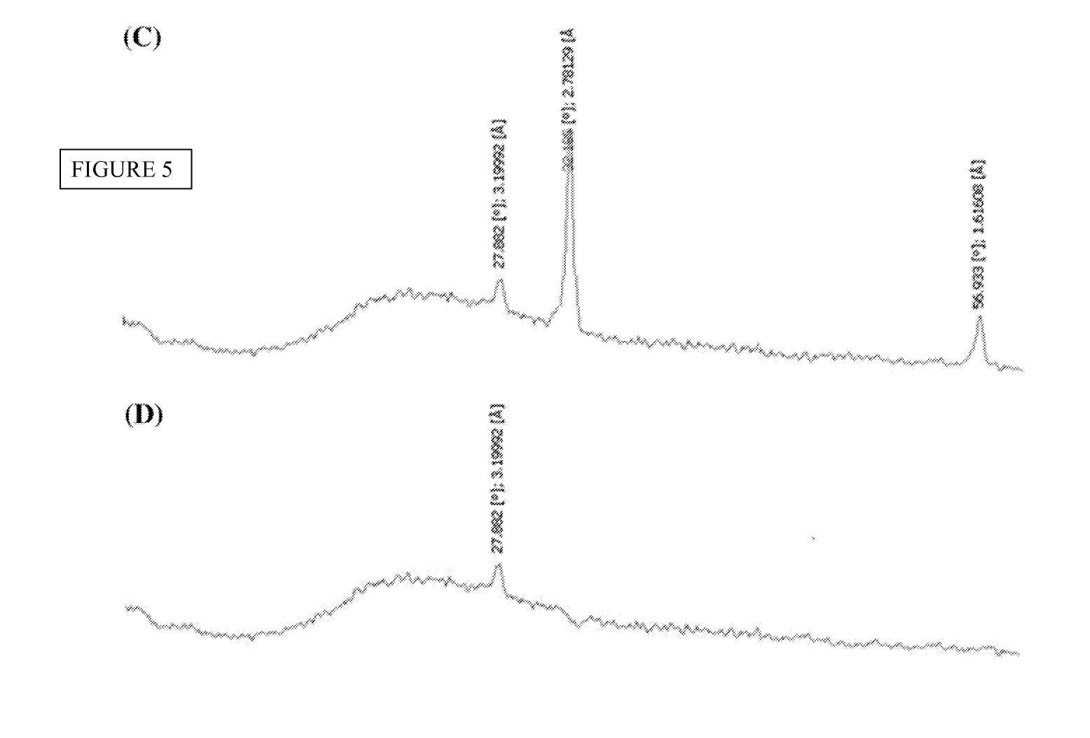

Crystallinity of CH-AT nano-conjugate: To identify the physical state and crystallinity of AT in polymeric conjugate, the XRD spectra of pure AT, CH, CH-AT conjugate and CH-AT nano-conjugate are presented in FIG. 5. As can be seen from FIG. 5, pure AT is highly crystalline. CH powder showed two major broad crystalline peaks at 2θ of around 9.5° and 19.7°, respectively, while the diffraction peaks of CH-AT conjugate were not recorded at the same position. The peak at 2θ of around 9.5° disappeared and instead new peaks at 2θ of 27.8°, 32.1° and 56.9° with low intensity could be observed. The reduction in crystalline peaks and formation of new peaks in CH-AT conjugate may be attributed to a polymorph structure transformation due to the attachment of CH to AT. In contrast to this, CH-AT nanoconjugate showed a broad amorphous peak. The possibility of shear-induced amorphous drug formation during the milling process could not be ruled out. Keck and Müller, Eur. J. Pharm. Biopharm., 62: 3-16 (2006); Kipp, Int. J. Pharm. 284: 109-122 (2004).

Solubility studies: The aqueous solubility of pure AT, CH-AT conjugate and CH-AT nano-conjugate was found to be 23.5, 589.2 and 2410.2 μg/mL, respectively. The solubility of AT was increased by approximately 25-fold after conjugation with CH. As expected, the solubility of CH-AT nano-conjugate was approximately 4-fold greater than the CH-AT conjugate, and nearly 100-fold higher than that of pure AT. This improved water solubility of AT for CH-AT nano-conjugate could be attributed to the collective effect of formation of water soluble conjugate, amorphous AT in CH-AT nano-conjugate, and reduced particle size which offer higher surface area for drug dissolution.

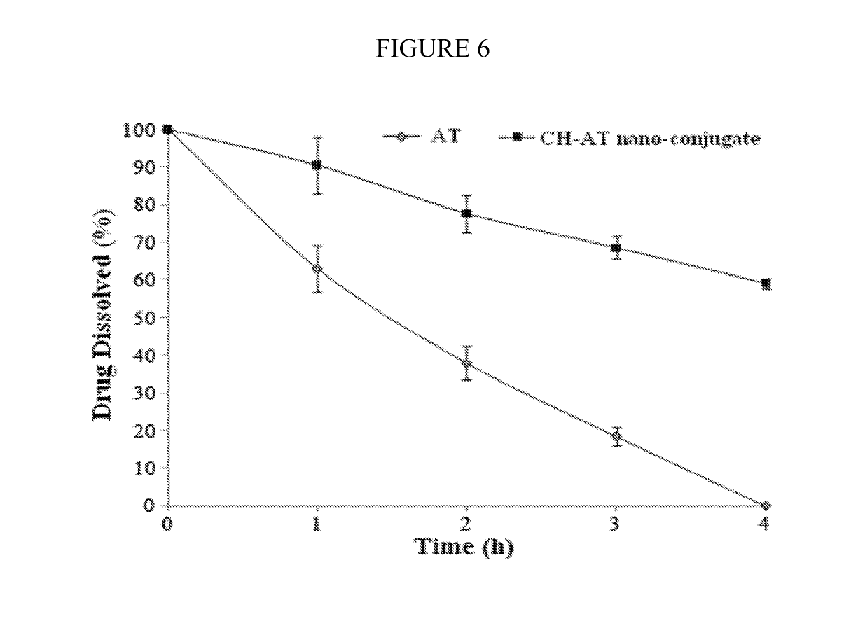

Acidic degradation kinetic studies: The study was performed to determine if CH-AT nano-conjugate would be able to prevent the acid-catalyzed degradation of AT. FIG. 6 shows the degradation kinetics of pure AT and CH-AT nano-conjugate. From FIG. 6 it was evident that for pure AT, complete drug degradation occurred at 4 h time point, whereas approximately 60% of AT was still remaining in case of CH-AT nano-conjugate. It can be anticipated that incomplete drug release from the CH matrix (FIG. 6) and presence of hydrophilic coating of CH over AT might be responsible for reduction in degradation of AT.

Evaluation of mucoadhesive properties of CH-AT nano-conjugate. The binding responses of pure AT and CH-AT nano-conjugate on intestinal membrane after 4 h were found to be 10.2% and 68.9%, respectively. The mucoadhesive nature of the CH-AT nano-conjugate was due to the presence of CH, which is known to be mucoadhesive. This result suggests clearly that CH-AT nano-conjugate retains mucoadhesiveness of parent CH. It is hypothesized that mucoadhesiveness observed for pure AT might be due to its hydrophobicity, resulting in enhanced interaction with the intestinal epithelium.

### Example 4

### In Vitro Release Studies

The purpose of this example was to characterize the in vitro release parameters of the nano-sized chitosan-statin conjugate prepared in Example 2.

In vitro release studies were performed using transparent gelatin capsules containing pure AT and the formulations (CH-AT conjugate and CH-AT nano-conjugate) equivalent to 100 mg of AT. Tests were performed employing United States Pharmacopeia (USP) paddle apparatus (Vankel apparatus, USA) using phosphate buffer (pH 7.4), and simulated gastric fluid (SGF, pH 1.2) at 37±0.1° C. for up to 72 h at a rotation speed of 50 rpm. At designated time points, 4 mL samples were withdrawn with replacement with equal volume of the fresh medium, filtered through 0.11 lm nylon syringe filter, appropriately diluted with methanol and assayed for drug concentration by UHPLC method as described below. Dissolution tests were performed in triplicate and the percentage of drug dissolved at different time intervals was estimated.

Results: To examine whether or not parent AT is released from the nanoconjugate, drug release experiments were carried out at 37° C. by incubation in SGF (pH 1.2) and phosphate buffer (pH 7.4). The release data clearly indicated that AT is released from the conjugate under physiological conditions (Table 2).

TABLE 2
In vitro release studies of CH-AT nanoconjugate.

% Drug dissolved

| Time (h) | SGF (pH 1.2) | Phosphate buffer (pH 7.4) |
|---|---|---|
| 0.5 | 4.7 ± 0.5 | 2.7 ± 0.3 |
| 1 | 10.5 ± 1.2 | 5.5 ± 0.8 |
| 2 | 21 ± 1.7 | 12 ± 1.3 |
| 4 | 72.5 ± 4.5 | 28.5 ± 1.8 |
| 6 | 100 ± 6.4 | 47.5 ± 3.1 |
| 8 | | 68 ± 4.8 |
| 10 | | 92.5 ± 6.2 |

Results represents mean values ± standard deviation, n = 3.

Complete release of AT was attained in SGF within 6 h, whereas the similar extent of AT release was observed within 10 h for phosphate buffer (92.5±6.2%). Approximately, 20% of AT was released upon incubation in SGF within 2 h, which amount would seem insubstantial when considering the transit time in stomach (1-2 h). These observations would be quite useful since acid catalyzed degradation of AT, which is responsible for variable bioavailability, would be significantly reduced.

### Example 5

### In Vivo Pharmacokinetic Studies

The purpose of this example was to evaluate the nano-sized Atorvastatin-chitosan conjugate in vivo pharmacokinetic studies.

For in vivo pharmacokinetics, two groups, each containing six female albino rats (0.18-0.22 kg) was used. After 12 h of fasting, the rats were allowed to administer 0.5 mL aqueous dispersion of AT, CH-AT conjugate and CH-AT nano-conjugate (equivalent to 10 mg/mL AT) using oral feeding sonde. Blood samples (0.2 mL) were withdrawn at pre-determined time intervals through the tail vein of rats in vacutainer tubes, vortexed to mix the contents and centrifuged at 5000 rpm for 20 min. The plasma was separated and stored at −20° C. until drug analysis was carried out using UHPLC method as described above. Data processing for calculating the pharmacokinetic parameters (PK) was done using Microsoft Excel software.

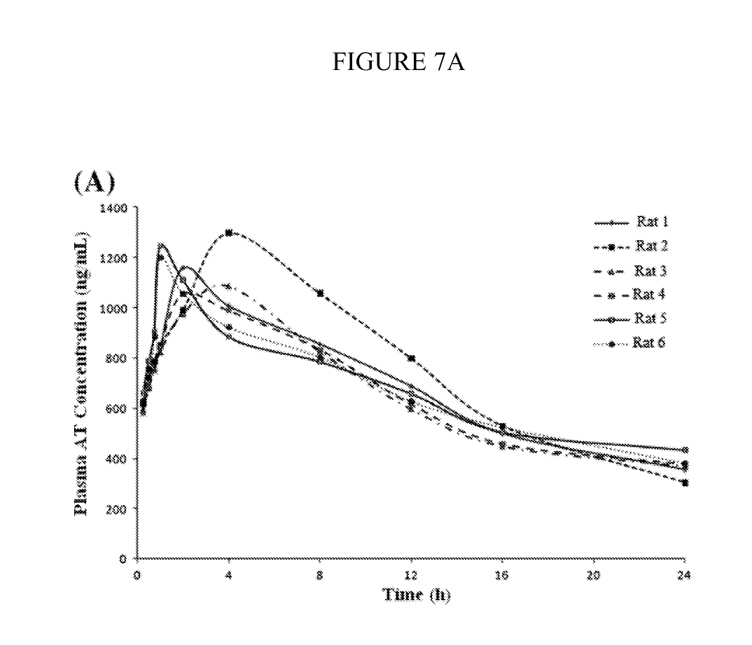

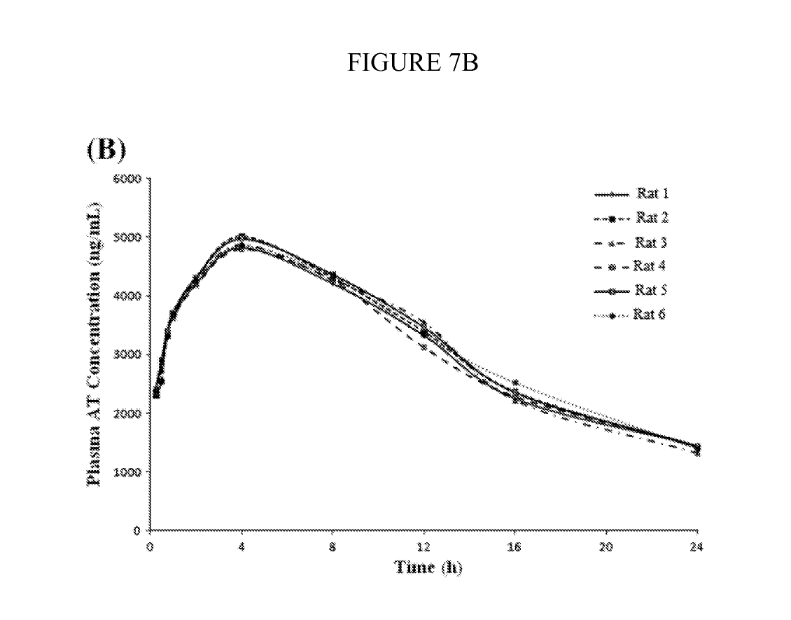

Pharmacokinetics after oral administration of CH-AT nanoconjugates to rats. FIGS. 7A and B depict the plot of AT concentration in plasma as a function of time individually for each rat in the group, after administration of AT suspension and CH-AT nano-conjugate solution, respectively. Plasma AT concentration vs. time plots obtained after oral administration of AT suspension to rats (FIG. 7A) clearly indicates that bioavailability is highly variable probably due to acid catalyzed degradation of AT or P-glycoprotein-mediated efflux. In contrast to this, the plasma AT concentration vs time plots obtained after oral administration of CH-AT nano-conjugate to rats exhibited nearly similar profile (FIG. 7B), indicating a reduction in variability in bioavailability. This could be either due to the prevention of acid catalyzed degradation by CH-AT nano-conjugate as demonstrated by acid degradation kinetic study or due to the ability of CH-AT nano-conjugate to bypass the P-glycoprotein-mediated efflux. as reported previously for oral delivery of paclitaxel-chitosan conjugate. Lee et al., J. Med. Chem. 51: 6442-6449 (2008). The relevant pharmacokinetic parameters are listed in Table 3.

TABLE 3
In vivo Pharmacokinetic studies of AT and CH-AT nanoconjugate.
Pharmacokinetic parameters

| Parameters | AT | CH-AT Nano-conjugate |
|---|---|---|
| AUC_{0→t} | 8240.6 | 37252.9 |
| (ng/mL h) | (2180.5) | (452.8) |
| AUC_{0→∞} | 11878.5 | 1047629.0 |
| (ng/mL h) | (2250.7) | (949.6) |
| Cₘₐₓ | 583.0 | 2574.0 |
| (ng/mL) | (55.5) | (95.4) |
| tₘₐₓ (h) | 2 | 4 |
| t_{1/2} (h) | 15.8 | 19.3 |

Results represents mean values (standard deviation), n = 6.

While AT suspension showed plasma half-life (t_{1/2}) of 15.8 h, CH-AT nano-conjugate group exhibited delayed t_{1/2} value of 19.3 h suggesting that AT is released from CH-AT nano-conjugate in a sustained manner over prolonged period of time. In addition, the Cₘₐₓ of the nano-conjugate (2574±95.4 ng/mL) was greater than that of AT suspension (583±55.5 ng/mL). As expected, a marked increment by 5-fold was observed in AUC_{0-∞} of CH-AT nano-conjugate as compared to AT suspension group. The oral bioavailability of AT from CH-AT nano-conjugate is the highest among others reported in the literatures to date. Kim et al., Int. J. Pharm., 359: 211-219 (2008); Kim et al., Eur. J. Pharm. Biopharm., 69: 454-465 (2008); Shen and Zhong, J. Pharm. Pharmacol., 58:1183-1191 (2006); Zhang et al., Int. J. Pharm. 374: 106-113 (2009).

This unprecedented high absorption may be attributed to enhanced solubility of amorphous AT in CH-AT nano-complex, the known ability of CH to be mucoadhesive and open tight junctions in intestinal epithelial cells. Furthermore, CH-AT nano-conjugate may also be able to bypass both P-glycoprotein-mediated efflux (displayed on intestinal epithelial cells) and cytochrome P450-mediated drug metabolism (hepatic clearance) as demonstrated previously for oral delivery of paclitaxel in the form of conjugate with chitosan. Lee et al., J. Med. Chem. 51: 6442-6449 (2008). The possible mechanism of drug release and bioavailability enhancement of AT through CH-AT nano-complex is depicted in FIG. 8.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A pharmaceutical composition comprising:
(a) a conjugate between chitosan and a drug selected from the group consisting of a chemotherapeutic agent, antibiotic, antifungal, and an asthma drug; and
(b) at least one pharmaceutically acceptable carrier,
wherein the chitosan-drug conjugate has a particle size of less than about 1000 nm.

2. The composition of claim 1, wherein:
(a) the chemotherapeutic agent is:
(i) selected from the group consisting of taxanes, alkylating agents, anti-metabolites, Topoisomerase inhibitors, and Cytotoxic antibiotics;
(ii) selected from the group consisting of paclitaxel, docetaxel, melphalan, chlorambucil, cyclophosphamide, mechlorethamine, uramustine, ifosfamide, carmustine, lomustine, streptozocin, busulfan, thiotepa, cisplatin, carboplatin, nedaplatin, oxaliplatin, satraplatin, triplatin, tetranitrate, procarbazine, altretamine, dacarbazine, mitozolomide, temozolomide, azathioprine, mercaptopurine, Azathioprine, Mercaptopurine, Thioguanine Fludarabine, Pentostatin, cladribine, 5-fluorouracil (5FU), Floxuridine (FUDR), Cytosine arabinoside (Cytarabine), 6-azauracil, methotrexate, trimethoprim, pyrimethamine, pemetrexed, raltitrexed, pemetrexed, Vincristine, Vinblastine, Vinorelbine, Vindesine, Etoposide, teniposide, camptothecins, irinotecan, topotecan, amsacrine, etoposide, etoposide phosphate, and teniposide, actinomycin, anthracyclines, doxorubicin, daunorubicin, valrubicin, idarubicin, epirubicin, bleomycin, plicamycin, and mitomycin; or
(iii) any combination thereof;
(b) the antibiotic is selected from the group consisting of almecillin, amdinocillin, amikacin, amoxicillin, amphomycin, amphotericin B, ampicillin, azacitidine, azaserine, azithromycin, azlocillin, aztreonam, bacampicillin, bacitracin, benzyl penicilloyl-polylysine, bleomycin, candicidin, capreomycin, carbenicillin, cefaclor, cefadroxil, cefamandole, cefazoline, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, chlortetracycline, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), dactinomycin, dalbavancin, dalfopristin, daptomycin, daunorubicin, demeclocycline, detorubicin, dicloxacillin, dihydrostreptomycin, dirithromycin, doxorubicin, doxycycline, epirubicin, erythromycin, eveminomycin, floxacillin, fosfomycin, fusidic acid, gemifloxacin, gentamycin, gramicidin, griseofulvin, hetacillin, idarubicin, imipenem, iseganan, ivermectin, kanamycin, laspartomycin, linezolid, linocomycin, loracarbef, magainin, meclocycline, meropenem, methacycline, methicillin, mezlocillin, minocycline, mitomycin, moenomycin, moxalactam, moxifloxacin, mycophenolic acid, nafcillin, natamycin, neomycin, netilmicin, niphimycin, nitrofurantoin, novobiocin, oleandomycin, oritavancin, oxacillin, oxytetracycline, paromomycin, penicillamine, penicillin G, penicillin V, phenethicillin, piperacillin, plicamycin, polymyxin B, pristinamycin, quinupristin, rifabutin, rifampin, rifamycin, rolitetracycline, sisomicin, spectrinomycin, streptomycin, streptozocin, sulbactam, sultamicillin, tacrolimus, tazobactam, teicoplanin, telithromycin, tetracycline, ticarcillin, tigecycline, tobramycin, troleandomycin, tunicamycin, tyrthricin, vancomycin, vidarabine, viomycin, virginiamcin, and rifampin;
(c) the antifungal is:
(i) selected from the group consisting of azoles, antimetabolites, allylamines, morpholine, glucan synthesis inhibitors, polyenes, benoxaborales, sodarin derivatives, and nikkomycins;
(ii) selected from the group consisting of Bifonazole, Clotrimazole, Econazole, Miconazole, Tioconazole, Fluconazole, Itraconazole, Ketoconazole, Pramiconazole, Ravuconazole, Posaconazole, Voriconazole, Flucytosine, Terbinafine, Naftidine, amorolfine, Caspofungin, Micafungin, Anidulafungin, Amphotericin B, Nystatin, pimaricin, AN2690, griseofulvin and ciclopirox; or
(iii) any combination thereof; or
(d) the asthma drug is:
(i) selected from the group consisting of inhaled corticosteroids, leukotriene modifiers, long-acting beta agonists (LABAs), theophylline, and oral corticosteroids;
(ii) selected from the group consisting of fluticasone, budesonide, mometasone, beclomethasone, and ciclesonide; montelukast, zafirlukast, zileuton, salmeterol, formoterol, albuterol, levalbuterol, pirbuterol, ipratropium, prednisone and methylprednisolone; or
(iii) any combination thereof.

3. A pharmaceutical composition comprising:
(a) a conjugate between chitosan and a statin; and
(b) at least one pharmaceutically acceptable carrier.

4. The pharmaceutical composition of claim 3, wherein the statin is not atorvastatin.

5. The composition of claim 3 additionally comprising a fenofibrate nanoemulsion, wherein the nanoemulsion comprises:
(a) fenofibrate;
(b) at least one solvent;
(c) at least one surfactant; and
(d) at least one oil.

6. The composition of any one of claims 3-5, wherein the statin is selected from the group consisting of atorvastatin, fluvastatin, lovastatin, pravastatin, pitavastatin, rosuvastatin, simvastatin, velostatin, fluindostatin, and rivastatin.

7. The composition of any one of claims 1-6, wherein:
(a) the conjugate is formed using amide coupling reaction between the amine groups of chitosan and an activated group of the statin or chemotherapeutic agent;
(b) the resultant conjugate comprises an amide linker that is cleaved under physiological conditions; or
(c) any combination thereof.

8. The composition of any one of claims 1-7, wherein the chitosan-drug conjugates:
(a) have an average particle size of less than about 1000 nm;
(b) have an average particle size selected from the group consisting of less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, and less than about 50 nm; or
(d) any combination thereof.

9. The composition of claim 8, wherein the nanosized chitosan-drug conjugates:
(a) demonstrate an increase in water solubility of the component drug as compared to a non-nanosized chitosan conjugate of the same drug, present at the same dosage;
(b) demonstrate an increase in bioavailability of the component drug as compared to a non-nanosized chitosan conjugate of the same drug, present at the same dosage;
(c) demonstrate an increase in mucoadhesion as compared to a non-nanosized chitosan conjugate dosage form of the same drug, present at the same dosage;
(d) prevent the degradation of the component drug in the acidic milieu of the stomach; or
(e) any combination thereof.

10. The composition of any one of claims 1-9, wherein:
(a) the Tₘₐₓ of the drug present in the chitosan-drug conjugate, when assayed in the plasma of a mammalian subject following administration, is less than the Tₘₐₓ for a conventional, non-chitosan nanosized conjugate form of the same drug, administered at the same dosage;
(b) the Cₘₐₓ of the drug present in the chitosan-drug conjugate, when assayed in the plasma of a mammalian subject following administration, is greater than the Cₘₐₓ for a conventional, non-chitosan nanosized conjugate form of the same drug, administered at the same dosage;
(c) the AUC of the drug present in the chitosan-drug conjugate, when assayed in the plasma of a mammalian subject following administration, is greater than the AUC for a non-chitosan nanosized conjugate form of the same drug, administered at the same dosage; or
(d) any combination thereof.

11. The composition of any one of claims 1-10, wherein:
(a) the pharmacokinetic profile of the drug present in the chitosan-drug conjugate is not substantially affected by the fed or fasted state of a subject ingesting the composition, when administered to a human;
(b) administration of the composition to a subject in a fasted state is bioequivalent to administration of the composition to a subject in a fed state; or
(c) any combination thereof.

12. The composition of any one of claims 1-11 formulated:
(a) into a dosage form for administration selected from the group consisting of oral, pulmonary, inhalation, intravenous, rectal, otic, opthalmic, colonic, parenteral, intracisternal, intravaginal, intraperitoneal, local, buccal, nasal, and topical administration;
(b) into a dosage form selected from the group consisting of liquid dispersions, gels, aerosols, ointments, creams, tablets, sachets and capsules;
(c) into an oral dosage form;
(d) into a dosage form selected from the group consisting of lyophilized formulations, fast melt formulations, controlled release formulations, delayed release formulations, extended release formulations, pulsatile release formulations, and mixed immediate release and controlled release formulations; or
(e) any combination thereof.

13. Use of a composition according to any one of claims 1-12 for the manufacture of a medicament.

14. The use of claim 13, wherein the medicament is useful in:
(a) treating or preventing dyslipidemia, hyperlipidemia, hypercholesterolemia, cardiovascular disorders, hypertriglyceridemia, coronary heart disease, peripheral vascular disease, symptomatic carotid artery disease), wherein the composition comprises a chitosan-statin conjugate;
(b) reducing LDL-C, total-C, triglycerides, and/or Apo B in adult patients with primary hypercholesterolemia or mixed dyslipidemia (Fredrickson Types IIa and IIb), wherein the composition comprises a chitosan-statin conjugate;
(c) treating adult patients with hypertriglyceridemia (Fredrickson Types IV and V hyperlipidemia), wherein the composition comprises a statin-chitosan conjugate;
(d) treating pancreatitis, wherein the composition comprises a chitosan-statin conjugate;
(e) treating restenosis, wherein the composition comprises a chitosan-statin conjugate;
(f) treating Alzheimer's disease, wherein the composition comprises a chitosan-statin conjugate;
(g) treating, preventing, or reducing the risk of a cancer, wherein the composition comprises a chitosan-chemotherapeutic agent conjugate;
(h) treating, preventing, or reducing the risk of a cancer, wherein the cancer is a solid tumor, and wherein the composition comprises a chitosan-chemotherapeutic agent conjugate;
(i) treating, preventing, or reducing the risk of a cancer, wherein the cancer is a hematopoietic disorder, and wherein the composition comprises a chitosan-chemotherapeutic agent conjugate;
(j) treating a microbial infection;
(k) treating a respiratory disease; or
(l) treating asthma.

15. A method of making a nanosized chitosan-drug conjugate, wherein the drug is a statin, chemotherapeutic agent, antibiotic, antifungal, or asthma drug, comprising:
(a) activating a chemical group of the statin, chemotherapeutic agent, antibiotic, antifungal, or asthma drug;
(b) covalently attaching the statin, chemotherapeutic agent, antibiotic, antifungal, or asthma drug to chitosan via an amide linker using an amide coupling reaction between amine groups of chitosan and the activated group of the drug to obtain a chitosan-drug conjugate; and
(c) homogenizing the chitosan-drug conjugate to reduce the particle size of the chitosan-drug conjugate to less than about 1000 nm.

16. The method of claim 15, wherein:
(a) the amide linker is cleaved under physiological conditions;
(b) the activated group is an activated carboxylic group;
(c) the homogenization process is a high pressure homogenization process;
(d) the chitosan-drug conjugates are lyophilized or spray dried prior to or after the homogenization process;
(e) the method further comprises adding a fenofibrate nanoemulsion to the chitosan-drug conjugate composition;
(f) the method further comprises adding a fenofibrate nanoemulsion to the chitosan-drug conjugate composition and the fenofibrate nanoemulsion is lyophilized or spray dried to form a powder prior to combining with chitosan-drug conjugate composition; or
(g) any combination thereof.

17. A method of delivering a composition according to claim 1 directly into the lungs of a subject, wherein:
(a) administration of the composition is by inhalation;
(b) the drug present in the composition is delivered at a dosage which is less than half of that required for oral or parenteral delivery of the same drug, to obtain the same therapeutic effect.
